(19) United States
(12) Patent Application Publication
(10) Pub. No.: US 2013/0130354 A1
(43) Pub. Date: May 23, 2013

(54) Novel genes and uses thereof, expression profile of colon, gastric and pancreatic cancer

(71) Applicants: Manuel Gidekel, Santiago (CL); Carolina Del Carmen Bizama Soto, Santiago (CL); Felipe Mario Benavente Muñoz, Santiago (CL); Hieda Ana Gutievez Moraga, Santiago (CL); Osvaldo Luis Podhajcer, Cludad de Buenos Aires (AR); Edgardo Enrique Salvatierra Colussi, Cludad de Buenos Aires (AR); Guiller Mo Daniel Mazzolini, Santiago (CL); Juan Carlos Roa, Temvco (CL)

(72) Inventors: Manuel Gidekel, Santiago (CL); Carolina Del Carmen Bizama Soto, Santiago (CL); Felipe Mario Benavente Muñoz, Santiago (CL); Hieda Ana Gutievez Moraga, Santiago (CL); Osvaldo Luis Podhajcer, Cludad de Buenos Aires (AR); Edgardo Enrique Salvatierra Colussi, Cludad de Buenos Aires (AR); Guiller Mo Daniel Mazzolini, Santiago (CL); Juan Carlos Roa, Temvco (CL)

(73) Assignee: CTI Salud S.A., Santiago (CL)

(21) Appl. No.: 13/136,760

(22) Filed: Aug. 10, 2011

Related U.S. Application Data
(60) Provisional application No. 61/404,141, filed on Sep. 28, 2010.
(60) Provisional application No. 61/404,141, filed on Sep. 28, 2010.

(51) Int. Cl. C12Q 1/68

(52) U.S. Cl. CPC C12Q 1/6886
USPC 435/219; 506/9; 435/375; 530/350; 530/351; 530/359; 530/399

(57) ABSTRACT

This invention provides information on differentially expressed genes in malignant tissue of gastric, colon and pancreatic adenocarcinomas as compared to their corresponding adjacent non-malignant tissues. These genes or their products can be used as targets in developing new strategies for the treatment and diagnosis of these gastrointestinal cancers.

This application claims priority under 35 U.S.C 119 (e) of U.S. Provisional 61/404,141, filed Sep. 28, 2010. The entire contents of the prior application U.S. Provisional 61/404,141 are incorporated herein by reference.

### BACKGROUND OF THE INVENTION

Gastrointestinal cancers rank second as a group in the most frequent neoplasias of men and women in the United States and are one of the most prevalent cancers in the world in general (Ajani, Curley et al. 2005). Within this group, colorectal cancer is the most frequent neoplasia, with more than one million new cases every year. It accounts for approximately 9.4% of all cases of cancer in the world. Likewise, there are more than 930,000 new cases of gastric cancer in the world every year and 230,000 cases of pancreatic cancer (WHO 2008). The most serious case is that of pancreatic cancer, where the 1-year survival rates are below 5% worldwide (Maitra, Kern et al. 2006). Together, these three neoplasias cause more than 1,700,000 deaths annually and the five-year survival rates are low. The reasons for the high mortality caused by these neoplasias is their inherent aggressiveness, the recurrence of local and distant metastasis, the inefficiency of current treatments and high resistance to antineoplastic drugs (Ajani, Curley et al. 2005). There is as yet no clear definition of the molecular basis of their aggresiveness.

The carcinogenic process is characterized by the generation of cells that are genetically and phenotypically different from their cell of origin. During this process, the tumor cell acquires new features that make it more resistant to treatment and more aggressive, coupled with an increased deregulation of molecules that will regulate, under normal conditions, processes required to maintain a balance between the cell and its neighborhood (Hanahan and Weinberg 2000). Among the molecules that are found to be deregulated most frequently in tumors are the matrix metalloproteinases, urokinase, E-cadherin, β-catenin and TGFβ, which are currently the subject of intense investigation aimed at blocking or regulating their activity (Mack and Marshall 2010). Identifying new molecular targets capable of being modulated by pharmacological or molecular agents is an area of great interest in cancer research (Benson, Chen et al. 2006).

One of the methodologies currently used to identify variations in the levels of gene expression in cancer tissues is the microarray technique based on cDNAs or oligonucleotides. These microarrays allow assessing gene expression of thousands of genes simultaneously; leading to the identification of tumor-associated genes whose differential expression as compared to normal tissue may play a leading role in tumor biology, and to determine, after experimental validation, whether or not they have the potential to become therapeutic targets (Bild, Yao et al. 2006).

### SUMMARY OF THE INVENTION

This invention identifies the global changes in gene expression profiles associated with gastric cancer, pancreatic cancer and colon cancer by comparing gene expression in the malignant tissue as such in comparison to paired adjacent non-malignant tissue.

Gene expression profiles were obtained from samples using the microarray technology. Genes involved in the cancer process were identified that may be used in developing new therapies for the treatment of these three gastrointestinal cancers.

This invention describes genes whose expression is elevated or reduced in samples from patients with gastric, colon and pancreatic cancer compared to samples of adjacent tissue diagnosed as non-malignant tissue. The information herein described not only contributes to a greater understanding of the tumor biology of these three cancers. It also provides alternatives for the development of new therapeutic strategies that may contribute to cancer therapy. These strategies may encompass: reducing the expression or interfering with the biological function of some of the genes described or restoring the expression of repressed or absent genes provided by the invention, in addition to tracking the condition of the disease, progression, toxicity, efficacy and metabolism of the drugs used in treatment.

The invention includes methods to diagnose for the presence or absence of the disease in the patient by detecting the levels of gene expression in a tissue sample of one or several genes where the expression of these genes is indicative of the presence of the disease.

In other respects, this invention provides a method for monitoring the efficacy of a treatment of a patient with gastric, colon or pancreatic cancer that includes the administration of a pharmaceutical compound or specific surgery procedure, by comparing the profile of the gene expression in blood samples containing tumor cells of the patient to the profile obtained in healthy cells or tissue. Some of the genes that must be included in the expression profile are listed in FIG. 3 to FIG. 7.

Moreover, this invention provides the method of treating a patient with gastric, colon or pancreatic cancer that includes any intervention, provided that intervention causes an alteration in the expression of at least one of the genes contained in FIG. 3 to FIG. 7. A gene expression profile is prepared from the patient's cell samples or tissue in comparison to samples from populations in which the original tissue is untreated.

Any of the methods described below may include detecting at least two genes from the stated tables. It is advisable to include several, if not all, in determining the presence or absence of the disease and in monitoring the progression or course of the chosen treatment.

### BRIEF DESCRIPTION OF FIGURES

FIG. 1 is a table that provides information about 30 patients from whom gastrointestinal cancer tissues and corresponding adjacent tissues were obtained. Histological grading of each cancer tissue was determined according to the classification of WHO. The metastases were represented as a ratio between the number of metastases in lymph nodes/total number of nodes examined.

FIG. 2 is a schematic representation of the subtractive hybridization (SSH)/microarray and regular microarray strategy used in this invention.

FIG. 3 is a table showing the genes differentially expressed in gastric cancer tissues as compared to corresponding adjacent non-cancerous tissues obtained by regular microarray strategy. Data for this table was performed in R statistical environment using Bioconductor packages. The table shows the number of genes differentially expressed (genes column), that were up-regulated and down-regulated. The identification of each gene (Symbol, Name and Entrez Gene_ID from Homo sapiens-database), their p-adjusted value by Benjamini & Hochberg, the average fold change and principal molecular functions and biological process that the gene is involved in. The data correspond to genes showing fold changes >1.2 and p-value <0.05.

FIG. 4 is a table showing the genes differentially expressed in gastric cancer tissues as compared to their corresponding adjacent non-cancerous tissues obtained by SSH/microarray strategy. Data for this table was performed in R statistical environment using Bioconductor packages. The table shows the number of genes differentially expressed (genes column), that were up-regulated and down-regulated. The identification of each gene (Symbol, Name and Entrez Gene_ID from Homo sapiens-database), their p-adjusted value by Benjamini & Hochberg, the average fold change and principal molecular functions and biological process that the gene is involved in. The data correspond to genes showing fold changes >1.5 and p-value <0.05.

FIG. 5 is a table showing the genes differentially expressed in colon cancer tissues as compared to corresponding adjacent non-cancerous tissues obtained by regular microarray strategy. Data for this table was performed in R statistical environment using Bioconductor packages. The table shows the number of genes differentially expressed (genes column), that were up-regulated and down-regulated. The identification of each gene (Symbol, Name and Entrez Gene_ID from Homo sapiens-database), their p-adjusted value by Benjamini & Hochberg, the average fold change and principal molecular functions and biological process that the gene is involved in. The data correspond to genes showing fold changes >1.2 and p-value <0.05.

FIG. 6 is a table showing the genes differentially expressed in colon cancer tissues as compared to corresponding adjacent non-cancerous tissues obtained by SSH/microarray strategy. Data for this table was performed in R statistical environment using Bioconductor packages. The table shows the number of genes differentially expressed (genes column), that were up-regulated and down-regulated. The identification of each gene (Symbol, Name and Entrez Gene_ID from Homo sapiens-database), their p-adjusted value by Benjamini & Hochberg, the average fold change and principal molecular functions and biological process that the gene is involved in. The data correspond to genes showing fold changes >1.3 and p-value <0.005.

FIG. 7 is a table showing the genes differentially expressed in pancreatic cancer tissues as compared to corresponding adjacent non-cancerous tissues obtained by regular microarray strategy. Data for this table was performed in R statistical environment using Bioconductor packages. The table shows the number of genes differentially expressed (genes column), that were up-regulated and down-regulated. The identification of each gene (Symbol, Name and Entrez Gene_ID from Homo sapiens-database), their p-adjusted value by Benjamini & Hochberg, the average fold change and principal molecular functions and biological process that the gene is involved in. The data correspond to genes showing fold changes >1.3 and p-value <0.005.

FIGS. 8a and 8b are two unsupervised hierarchical clustering analysis performed on gastric cancer samples and corresponding adjacent tissue. (a) analysis was performed with 119 genes differentially expressed using the regular microarray platform. (b) analysis was performed with 149 genes differentially expressed using the SSH/microarray platform. Group A: consists of gastric adenocarcinoma. Group B: consists of non-cancerous gastric tissue, each tissue sample being labeled with a unique number followed by A (adjacent normal tissue), or T (tumor tissue). Columns identify the patient sample, rows correspond to the gene name. A scale bar representing the relationship between the color intensity and gene expression log ratios is also shown. The levels of gene expression are color-coded from green (down regulated) to red (up regulated). The missing value genes in array are grey, near-zero values are in black.

FIGS. 9a and 9b are two unsupervised hierarchical clustering analysis performed on colon cancer samples and corresponding adjacent tissue. (a) analysis was performed with 108 genes differentially expressed using the regular microarray platform. (b) analysis was performed with 72 genes differentially expressed using the SSH/microarray platform. Group A: consists of colorectal adenocarcinoma. Group B: consists of non-cancerous colorectal tissue, each tissue sample being labeled with a unique number followed by A (adjacent normal tissue), or T (tumor tissue). Columns identify the patient sample, rows correspond to the gene name. A scale bar representing the relationship between the color intensity and gene expression log ratios is also shown. The levels of gene expression are color-coded from green (down regulated) to red (up regulated). The missing value genes in array are grey, near-zero values are in black.

FIG. 10 is an unsupervised hierarchical clustering. Analysis was performed on 450 genes differentially expressed in pancreatic cancer samples and the corresponding adjacent tissue. Group A: Includes pancreatic adenocarcinomas. Group B: Includes the non-cancerous adjacent tissue, each tissue sample being labeled with a unique number followed by A (adjacent normal tissue), or T (tumor tissue). Columns correspond with tissue samples, rows correspond to gene names. A scale bar representing the relationship between the color intensity and gene expression log ratios is also shown. The levels of gene expression are color-coded from green (down regulated) to red (up regulated).

FIG. 11a and 11b are a series of charts that show the RT-PCR validation of microarray data in gastric tissues. (a) The real time PCR quantification data of 25 genes of the regular microarray are shown. (b) Real-time PCR quantification data of 18 genes of the SSH/microarray is also shown. “Tumor” represents cancerous tissue and “Adjacent” represent the corresponding adjacent non-cancerous tissue. PCR reactions were performed in duplicate and the results were normalized using three genes: “QARS” Glutaminyl-tRNA synthetase, “POL2R2” polymerase (RNA) II polypeptide L and “TFCP2” Transcription factor CP2. Data are presented as the fold change in gene expression in cancer and adjacent noncancerous tissue relative to an inter-run calibrator. Statistical analysis and significance (one tail paired t-test), p-value summary: * <0.05; ** <0.01; *** <0.001, ns: no significant.

FIGS. 12a and 12b are a series of charts that show the RT-PCR validation of microarray data in colon tissues. (a) The real time PCR quantification data of 25 genes of the regular microarray are shown. (b) Real-time PCR quantification data of 20 genes of the SSH/microarray is also shown. “Tumor” represents cancerous tissue and “Adjacent” represent the corresponding adjacent noncancerous tissue. For each point, PCR reactions were performed in duplicate and the results were normalized using three genes: “TBP” TATA box binding protein, “POL2R2” polymerase (RNA) II polypeptide L and “TFCP2” Transcription factor CP2. Data are presented as the fold change in target gene expression in cancer and adjacent noncancerous tissue and relative to an inter-run calibrator. Statistical analysis and significance (one tail paired t-test), p-value summary: * <0.05; ** <0.01; *** <0.001, ns: no significant.

FIG. 13 are a series of charts that show the RT-PCR validation of microarray data. The real time PCR quantification data of 16 genes of the regular microarray is also shown. “Tumor” represents cancerous tissue and “Adjacent” represent the corresponding adjacent noncancerous tissue. For each point, PCR reactions were performed in duplicate and the results were normalized using three genes: “QARS” Glutaminyl-tRNA synthetase, “TBP” TATA box binding protein and “UBE2D” Ubiquitin-conjugating enzyme E2D-2. The data are presented as relative gene expression values in cancer and adjacent noncancerous tissue and relative to an inter-run calibrator. Statistical analysis and significance (one tail paired t-test), p-value summary: * <0.05; ** <0.01; *** <0.001, ns: no significant.

FIG. 14 is a table showing a list genes that encode secreted proteins obtained from the differentially expressed genes. The search of secreted proteins was performed in Gene Ontology (GO) using the DAVID bioinformatics resource.

FIGS. 15a and 15b are a series of charts that show the efficiency of the six shiRNA studied to downregulate the expression levels of selected genes in two gastric carcinoma cell lines, AGS and MKN45, colon carcinoma cells lines, HT29 and LoVo. RCC2, DUSP14 and NEK2 mRNA levels were monitored by qRT-PCR 48 hours after siRNA transfection. Each assay was performed in duplicate. In AGS and MKN45 cell lines, the transcripts level was normalized against POLR2L, QARS and TFCP2. For HT-29 and LoVo cells, POLR2L, TBP, TFCP2 transcript levels were used as normalizers. Data are expressed as the proportion of mRNA levels using the specific siRNA compared to the AllStars Negative control siRNA. Average percentages of mRNA depletion are indicated.

FIG. 16 In vitro migration of gastric cancer cells. Gastric adenocarcinoma cells (AGS) transfected with each specific siRNA were placed in the upper wells of a 48-well micro chemotaxis chamber and F12K medium with 10% FBS was placed in the lower chamber. After 48 hours, migrated cells were stained and counted. Upper: microphotograph showing migrated cells. Lower: migration capacity of cells transfected with each siRNA. Columns show the mean number of cells harvested from three experiments done in triplicate (bars show±SEM, *** p<0.001).

FIG. 17 In Vitro migration activity of colon cancer cells. HT29 cells transfected with each siRNA were placed in the upper wells of a 48-well micro chemotaxis chamber and complete medium with 10% FBS was placed in the lower chamber. After 24 hours in culture, the migrated cells were stained and counted. Migration activity of cells transfected with each siRNA. Columns show the mean number of cells harvested from two experiments done in triplicate (bars show±SEM).

FIG. 18 JPH1 expression in human colon carcinoma samples obtained from paraffin blocks. A) Complete absence of positive staining in normal colonic mucosa. B) Intense staining in invading colon cancer cells.

FIG. 19. PMEPA1 expression in pancreatic adenocarcinoma samples obtained from paraffin blocks. A) pancreatic ductal cells with preserved morphology and polarity showed faint PMEPA1 staining; B) pancreatic ductal cells with altered morphology and loss of polarity showed moderate intracytoplasmic PMEPA1 expression.

FIG. 20. PMEPA1 expression in normal pancreatic ducts. A) Normal ductal cells with preserved polarity and normal morphology showed no positive staining.

### MATERIALS AND METHODS

### Clinical Samples and Cell Lines

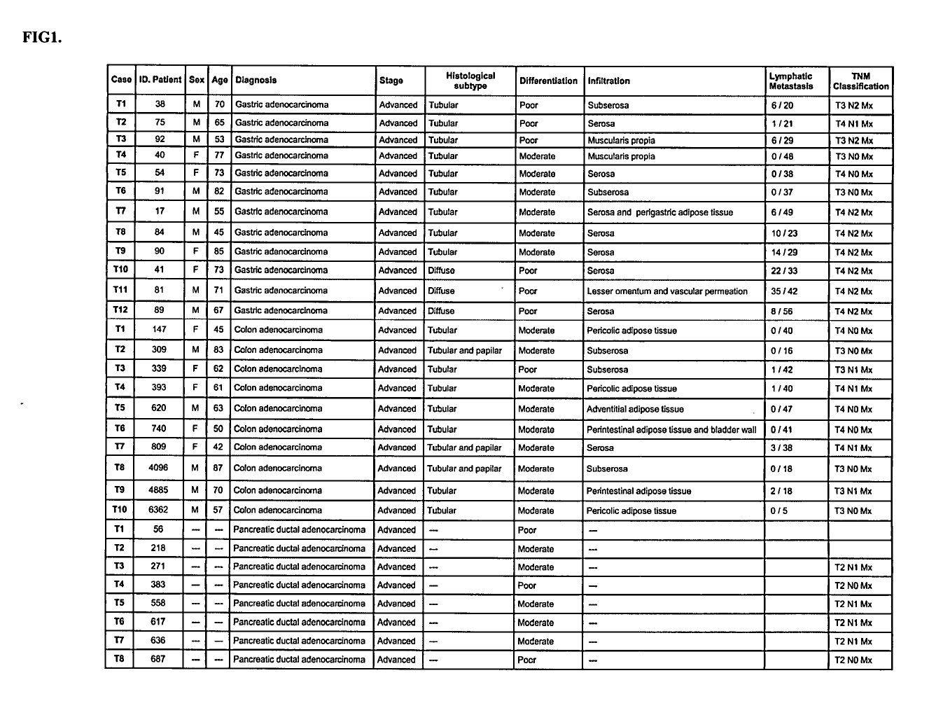

Samples were obtained from the Chile Tumor Bank and the Biobank of Universidad de la Frontera. Cancer tissue and the corresponding adjacent non-cancerous tissue were collected through an informed consent from 12 gastric adenocarcinoma patients, 10 colon adenocarcinoma patients and 8 pancreas adenocarcinoma patients who did not receive therapy prior to surgery. All tissues studied were submitted to histological analysis to confirm the presence of malignancy. FIG. 1 summarizes the characteristics of patient samples. Tissues were frozen in liquid nitrogen and stored at −90° C. until use.

The gastric adenocarcinoma cell lines (SNU-1, SNU-16, KatoIII, NCI-N87, MKN45, AGS), colon adenocarcinoma cell lines (HT-29, LoVo, Caco2, SW480, DLD-1, T89) and pancreas adenocarcinoma cell lines (SW1990, HS766T, BxPc3, MiaPaCa2, Panc-1, Capan-1) were obtained from the American Type Culture Collection (ATCC, Rockville, Md., USA). The cells were kept in their growth medium (recommended by ATCC), supplemented by 10%-20% characterized fetal bovine serum FBS (Hyclone, Logan, Utah, USA), and 100 U/ml of penicillin, plus 100 mg/mL of streptomycin (Gibco BRL, Gaithersburgh, Md., USA). They were grown under humidity conditions at 37° C. and 5% CO₂.

### RNA Extraction

The RNA from tissue and cells was extracted using Trizol (Invitrogen, Carlsbad, Calif., USA), following the supplier's instructions. From 50 to 250 mg of each tissue were pulverized with liquid nitrogen and 1 ml of Trizol. Once the cell lines reached 80% to 95% confluence in a 10 mm dish, they were washed twice with 1% PBS and resuspended in 1 ml of Trizol. The RNA pellet was resuspended in 30-100 ul of nuclease-free water, quantified and analyzed by electrophoresis in 1.2% agarose gel. Then 10 ug of each RNA was subjected to enzymatic digestion with buffer containing 2 U DNAse I (Ambion Inc, Austin Tx, USA), 40 U of ribonuclease inhibitor (RNAaseOUT, Invitrogen, Carlsbad, Calif., USA) in a total of 50 ul for 30 minutes at 37° C. At the end, RNA was brought to a volume of 100 ul of water and purified with the RNAeasy kit (QIAGEN, Hilden, Germany), following the supplier's protocol. Finally, the RNA was eluted from the column with 30 ul of water and the quantity and purity of RNA determined using UV spectrophotometry (NanoDrop Technologies, USA).

The mRNAs from normal gastric, colon and pancreas tissue was obtained from commercial sources (Clontech, Palo Alto, Calif., USA). These RNAs were obtained from patients with no history of premalignant lesions.

### Suppression Subtractive Hybridization (SSH)

The synthesis and amplification of cDNA prior to subtraction was done with the Clontech Super SMART PCR cDNA Synthesis kit (Clontech, Palo Alto, Calif., USA), following the supplier's protocol. 1 ug of RNA from each sample was used for the synthesis of the first strand in 50 ul, which was incubated with 7 ul of 3′ SMART CDS Primer IIA (12 uM) and 7 ul SMART IIA oligonucleotide (12 uM) for 2 minutes at 65° C. Then 20 ul of 5× First-Strand Buffer, 2 ul DTT (100 mM), 10 ul of 50× dNTP (10 mM), 2.5 ul of RNaseOUT (40 U/ul), 2 ul of SuperScript III Reverse Transcriptase 200 U/ul and 5.5 ul of sterile deionized water (ADESI) were added to the reaction and incubated for 90 minutes at 46° C. and 15 minutes a 70° C. The simple strand cDNA was then purified using the NucleoSpin Extract II kit (Macherey-Nagel, Düren, Germany) and eluted in 80 ul. In order to identify the number of optimal cycles required to begin suppression subtractive hybridization, the cDNA was amplified by long-distance PCR (LD-PCR) separately in 15, 18, 21, 24 and 27 cycles (Diachenko, Ledesma et al. 1996) and the PCR product was checked by electrophoresis in 1.2% agarose gel. The PCR was done by mixing all of the cDNA with 172 ul of deionized water, 30 ul of PCR Advantage 2 Buffer, 6 ul of dNTPs (10 mM), 6 ul of 5′PCR Primer IIA and 6 ul of Advantage 2 Polymerase Mix. The heat cycle was 95° C. for 1 minute and X cycles (optimal cycle) of 95° C. for 5 seconds, 65° C. for 5 seconds, and 68° C. for 6 minutes. 10 mg of total human placenta RNA was used for reaction control. CROMA-SPIN 1000 columns (Clontech, Palo Alto, Calif., USA) were used to purify the cDNA after the PCR.

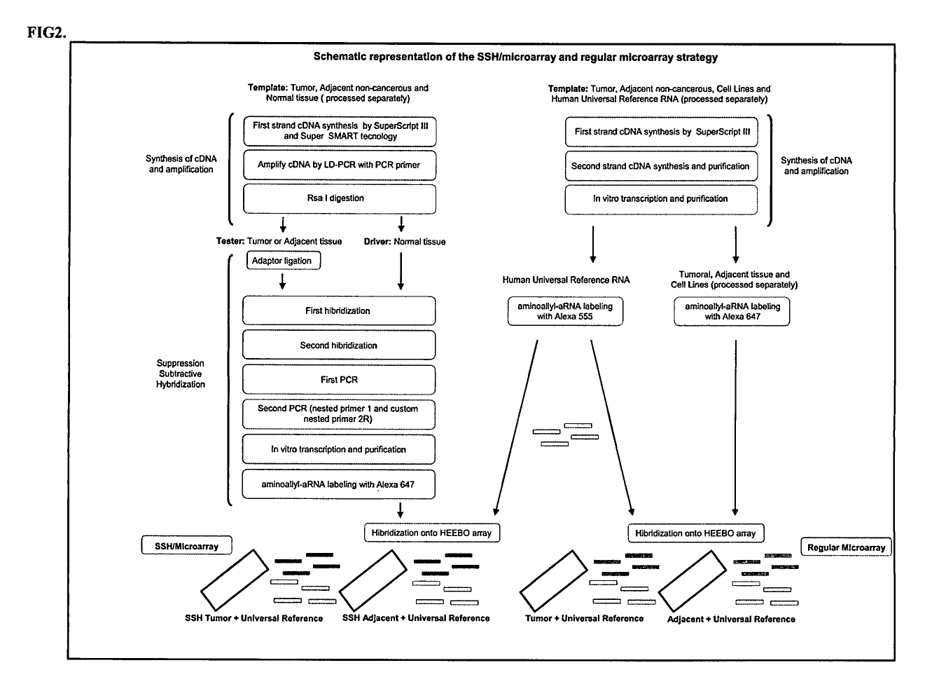

SSH was done following the protocol of the Clontech PCR-Select cDNA subtraction kit, with some modifications. During the secondary PCR, the nested 2R divider was replaced by the modified 2R nested sequence 5′-CTAATACGACTCACTATAGGGCTCGAGCGGCC-3′, which has a T7 promoter site that permitted the in vitro transcription of the subtractive amplicon and subsequent hybridization of the microarray. As an experimental design, a forward substraction was used while the tumor sample and corresponding adjacent non-cancerous tissue were used separately as a tester. cDNA of normal tissue was used for both samples as a driver (FIG. 2)

All of the cDNA from the tester and driver samples and control cDNA were digested individually in a total of 360 ul with 15 U of Rsa1, a restriction enzyme that recognizes four base pairs (5″-GTAC-3′) and leaves blunt ends on the digested DNA. Then cDNA of the tester sample was divided into two aliquots of 120 ng each and ligated separately to the adapters 1 and 2R, which resulted in two cDNA tester populations. The efficiency of the ligation reaction was checked using the PCR primer 1 and 3′ GAPDH dividers. During the first hybridization, a small amount of ligated tester cDNA was mixed separately with each adapter and the cDNA driver (450 ng), heat denatured and hybridized for 8 hours at 68° C. Two samples of this initial hybridization were then combined and hybridized with the denatured driver cDNA (300 ng) overnight at 68° C. The PCR amplification of the specific tester sequences was done using the PCR 1 divider. Finally, a second nested PCR was done using the PCR primer 1 nested dividers and the modified 2R nested PCR to reduce the levels of unspecific amplification and enrich the differentially expressed sequences. The conditions of the first PCR were as follows: incubation at 75° C. for 5 minutes, followed by a total of 27 cycles at 94° C. for 30 seconds, at 66° C. for 30 seconds and at 72° C. for 1.5 minutes. The secondary PCR was put through 10-12 cycles at an annealing temperature of 68° C.

The efficiency of the subtraction was checked, confirming the reduction of normalized genes in each of the samples subtracted by real time PCR.

### Microarray Labeling and Hybridization

The Human Exonic Evidence Based Oligonucleotide (HEEBO)

48.5K glass was used for hybridization (Microarray Inc, Huntsville, USA), developed by Stanford University and Illumina. These slides comprised one set of 39,000 probes, which encode for exon and alternative splicing sequences.

An indirect design was used for the hybridization of the microarray, where each aRNA from the regular strategy or the substractive amplicon labeled with Alexa 647, was hybridized against an aRNA from a Universal Reference labeled with Alexa 555 (Human Universal Reference, Clontech, Palo Alto, Calif., USA).

Amplification and labeling for the regular microarray strategy were done using the SuperScript Indirect RNA Amplification System (Invitrogen, Carlsbad, Calif., USA), following the supplier's instructions. 1 ug of total RNA from malignant, adjacent and normal samples and cell lines was used as a template for each reaction. The PCR products used for substraction were purified for SSW microarray using the E.Z.N.A. Cycle-Pure kit (Omega Bio-tek, Norcross, Ga., USA) and 300-500 ng of purified product were subsequently used as a template for the in vitro transcription.

The hybridization probe consisted of 60 pmoles of universal reference aRNA labeled with Alexa Fluor 555 and 60 pmoles of aRNA in an experimental condition labeled with Alexa Fluor 647 in a final volume of 50 ul with 1× hybridization buffer (SSC 5×, formamide at 50%, SDS 0.1%, 0.01% salmon sperm DNA) (except for the colon sample A5: 58 pmol of Alexa Fluor 555 with 46.4 pmol of Alexa Fluor 647). This mix was incubated for 2 minutes at 95° C. and applied to the slides after prehybridizing with SSC 5×, SDS 0.1% and 0.1% BSA for 30 minutes at 50° C. The hybridization was done in a wet chamber with SSC 2× (InSlide Out, Boekel Scientific, Pennsylvania, Pa., USA) for 16 hours at 42° C. After the hybridization, slides were washed once for 5 minutes at 42° C. with SSC 2×, SDS 0.1% and 5 min at room temperature with SSC 0.1×, SDS 0.1% and twice for 1 minute with SSC 0.1×. Finally, slides were dried by centrifugation and scanned in a ScanArray Gx (PerkinElmer, Wellesley, Mass., USA).

### Data Analysis

The signal intensity of the slides was quantified using the SpotReader Software (Niles Scientific, USA). The “orange pack 1” standard and an adaptive elliptical circle were used to grid the image. The artifacts inside the glass were eliminated manually and not used in the analysis.

The data were normalized and differential genes were obtained for the regular microarray colon strategy using the CarmaWeb server: comprehensive R- and bioconductor-based web service for microarray data analysis (https://carmaweb.genome.tugraz.at). The saturated and low-intensity spots were eliminated automatically before analysis. The background was subtracted using the “minimum” method. The “printipploess” method was used to normalize inside the slides while the “Gquantile” method was used to normalize intensities between slides. The genes differentially expressed between malignant samples and adjacent tissues were obtained using the Limma package for the gastric data and SAM for colon data. In both cases, the statistical test was moderated and paired and applied to the normalized M values. Finally, the Benjamini & Hochberg (BH) method was used to adjust the p-value.

The measured signal intensities for both SSH and Regular microarray gastric and SSH microarray colon approaches, were normalized [Smyth et al. 2003] using the free available statistical environment “R” (www.r-project.org) by using the bioconductor package “limma” [Smyth et al, 2003]. In all cases the spots intensities were background substracted (minimum method from limma package) and nonlinear intensity dependence in each array eliminated by “printiploess” method (Yang, Dudoit et al. 2002). Then, a median and scale correction (when appropriate) were applied for between array normalization yielding the expression matrix M^{GxA} where G is the number of genes in the array and A the number of arrays in the experiment. In order to identify differentially expressed genes between tumor and adjacent samples the following linear model where applied M_{gts}=(μ_{g}γ_{gs})+T_{gt}+ε_{gts}

where M_{gts} refers to the normalized expression value for gene “g”, in subject “s” under tissue type (tumor/adjacent) “t”, then μ_{g} is the overall mean for gene “g”, γ_{gs}˜N(0,σ_{γ}^{z}) is a random term accounting for subject correlation (the tumor and adjacent measures are paired), T is the difference in mean between tumor and adjacent sample (the main effect of interest) and ε˜N(0,σ^{z}) a random error term. Then, those genes for which the term T_{gt}≠0 is statistically different from zero where identified as differentially expressed.

Differentially expressed genes were grouped by unsupervised hierarchical clustering and visualized through heatmaps using the free available MeV program: multiExperiments Viewer, which is part of the TM4 Microarray Software Suite (http://www.tm4.org/mev/). The Euclidian distance was used as the standard for metric similarity.

Synthesis of cDNA and Quantitative Real Time PCR

In order to confirm the microarray data, differentially expressed genes were selected from the list of genes obtained by the SSH/microarray and the regular microarray strategy and the gene expression differences between malignant samples and tumor-adjacent tissue was compared using quantitative real-time PCR (qRT-PCR). The AffinityScript qRT-PCR commercially available kit was utilized for the cDNA synthesis (Stratagene, Cedar Creek, Tex., USA), following manufacturer's specifications. The reverse transcription using 1 ug of RNA and a mix of oligo dT (170 ng) and random primers (30 ng) was used as initiators in a total of 20 ul. The mix was incubated for 5 minutes at 25° C., 45 minutes at 42° C. and 5 minutes at 95° C. The PCR reaction took place in a total of 20 ul, using a template made of 2 ul of cDNA previously diluted to ⅕- 1/10 and a mix of 0.5 ul of dividers (10 uM), 10 ul Brilliant II SYBR Green (Stratagene, Cedar Creek, Tex., USA) and 7.5 ul of deionized water. The PCR was quantified in a Stratagene Mx3000p (Stratagene, Cedar Creek, Tex., USA), using the following heat cycle: 10 minutes at 95° C. and 40 cycles of 15 seconds at 95° C., 15 seconds at 60° C., 15 seconds at 72° C.

Three normalizing genes were used per tissue studied, QARS, POL2R2, TFCP2 for gastric cancer, POLR2L, TFCP2, TBP for colon cancer, and QARS, TBP, UBE2D2 for pancreatic cancer. These genes were selected since a small variation was observed between samples (tumor, adjacent and cell lines) using the GeNorm software (http://medgen.ugent.be/˜jvdesomp/genorm/). The dividers were designed using the amplifX 1.5.4 software and Primer3 and the efficiency of each was determined by the LinregPCR program (Ramakers, Ruijter et al. 2003).

The PCR data were analyzed with Stratagene MxPro software. Statistical significance between the two studied groups (malignant and the corresponding adjacent tissue) was determined with the GraphPad Prism 5.00 software for Windows (GraphPad Software Inc., San Diego, USA). A one-tailed paired Student's t-test was applied and p-value <0.05 was chosen as a significant value.

Transfection with siRNA

200,000 cells were placed in their respective growth medium in 6-well dishes at 10% FBS for 24 hours at 37° C. under a 5% CO₂ atmosphere. The cells were then washed once with PBS 1× and 900 ul medium was added with no serum or antibiotics. The cells were then transfected during 6 hours using 2.0-2.5 ul of Lipofectamine 2000 (Invitrogen, Carlsbad, Calif., USA) and an optimized concentration of siRNA for each cell line (5 nM for gastric cell lines and 1.67 nM for colon lines) in a final volume of 100 ul. After the incubation time, the transfection medium was removed and replaced by the growth medium, supplemented with 10% FBS (Hyclone., Logan, Utah, USA) and 100 U/mL of Penicillin, plus 100 μg/mL of Streptomycin (Gibco BRL, Gaithersburgh, Md., USA).

Each siRNA and the AllStars Negative control siRNA were obtained from QIAGEN (QIAGEN, Hilden, Germany). The oligonucleotide sequences were the following: PPAT (5′-UUCAGUUAUCGCACUUUGGTA-3′, cat. no SI00041552), JPH1 (5′-UUUAGCAUCUACACCUUCCTG-3′, cat. no. SI00449883), GTPBP4 (5′-UUCAUAUCAAACACGUGUCTA-3′, cat. no. SI00099547), DUSP14 (5′-UAACAGCUAAAUAAUAAGGGT-3′, cat. no. SI02659055), NEK2 (5′-UUAAGUUUAACAUCCUCGUAA-3′, cat. no. SI00605647), RCC2 (5′-UGCGGUAAGCAGCUUGCUGTT-3′, cat. no. SI02655569).

### Migration Assay

A 48-well chemotaxis chamber was used for this assay (Neuro Probe, Cabin. John, Md), with polycarbonate membranes containing 8 μm diameter pores (Neuro Probe, Cabin. John, Md, cat no. PFB8, 866-417-0014) and embedded in 0.1% gelatin (Sigma, St. Louis, Mo., cat. no. G2500). At the end of the incubation time (24-48 hours), cells were released with trypsin 0.05% EDTA 1 mM (Gibco, cat. no. 25300-054), resuspended in the respective medium with no serum and counted in a Neubauer chamber. A total of 15,000-20,000 cells were loaded in each well on the upper well of the chamber in a total of 50 ul, while in the lower phase growth medium containing 10% FBS was used as the chemoattractant. The assay was performed at 37° C. and 5% CO₂ for 7 to 12 hours. After that period, the membranes were fixed with absolute methanol, transferred to PBS 1× and the cells of the upper part of the membrane (that did not migrate) were removed using double-edged metal blades (super chromium, Schick, Germany). Finally, the membranes were washed with PBS, stained during 30 minutes with Giemsa solution (Azur-Eosin-Methylene blue Solution for Microscopy, Merck KgaA, 64271 Darmstadt, Germany) at 10% (diluted 1:10 in PBS 1×, pH.7.3), and visualized under a microscope. Each assay was done in triplicate and repeated at least 2 times. A negative migration control with no chemoattractant was included in all cases (non-serum medium).

Immunohistochemical analysis was performed on 6 samples of human colon carcinoma, 2 pancreatic ductal adenocarcinoma and 1 normal pancreatic sample. Representative pieces of adjacent and malignant tissues were fixed by immersion in neutral 10% buffered formalin (pH 6.8-7.2), dehydrated in serial alcohol dilutions, clarified with xylene and embedded in paraffin blocks. Then sections of 4 μm were obtained using a rotation microtome (RM 2245, Leica Nussloch, Germany) and mounted on silanized slides. Sections were incubated at 60° C. for 30 minutes, deparaffinized in xylene and rehydrated in serial ethanol dilutions. Antigen retrieval was performed in buffer citrate 10 mM (pH 6.0) inside a steamer for 10 minutes. To eliminate endogenous peroxidase activity, slides were incubated with an aqueous solution of hydrogen peroxide 3% for 10 minutes at room temperature and washed with TBS-Tween buffer 0.1% (TBS-T). Protein blocking was carried out with protein blocking solution for 15 minutes at room temperature (DAKO, # X0909). Then, sections were incubated with 100 μL of each primary antibody, properly diluted with antibody diluent (DAKO, # S3022), for 40 minutes at room temperature in a humidified chamber. For each antibody, a standardized dilution was used. For RCC2 antibody (Abcam, ab70788) we used a 1:500 dilution and for DUSP14 (Abnova, PAB4143) and Nek2 (Abcam, ab55550) antibodies, a 1:50 dilution was used. After incubation, the sections were washed with TBS-T buffer and incubated with 50 μL of HRP polymer secondary antibody conjugated for 15 minutes at room temperature in a humidified chamber (Super Picture Polymer, Invitrogen Cat No. 87 8963). Subsequently, the sections were washed with TBS-T and incubated with 50 μL of Substrate (Nova Red Vector Lab # SK-4800) monitoring the reaction under a microscope. Sections were counterstained with Mayer's hematoxylin and alkalinized with a saturated lithium carbonate solution. Finally, the sections were dehydrated through serial alcohol dilutions, placed in xylene and mounted.

### DETAILED DESCRIPTION OF THE INVENTION

Various biological functions are linked to changes in the expression levels of several genes through transcriptional control (such as through the control of initiation, RNA precursors, RNA processing, etc.) and/or translational control. For example, fundamental biological processes such as the cell cycle, differentiation and cell death are characterized by the variation in the levels of expressions of different groups of genes.

These changes are also related to pathogenesis. For example, the lack of a functional expression of tumor-suppressing genes and/or the overexpression of oncogenes or protoncogenes may lead to tumorigenesis or hyperplastic cell growth (Marshall 1991; Weinberg 1991).

Monitoring the changes in gene expression can show how a disease or treatment progresses.

In this disclosure, we have examined samples of malignant tissues from human tumor resections of gastric, colon and pancreatic malignancies. We analyzed changes in the global gene expression associated with each of the pathologies. The protocol used is depicted in FIG. 2.

These global changes in gene expression, also called expression profiles, provide markers useful in diagnosis and markers that can be used to monitor the stage, progression of the disease, toxicity, efficacy and metabolisms of the drugs used.

The present invention provides composition and methods to detect genes that can be expressed differentially, depending on the cellular stage, such as cancerous versus non-cancerous adjacent tissue. In this disclosure, the use of the expression “detection of genes expression levels” includes methods of quantifying gene expression and methods of determining whether a gene is expressed in Differentially expressed genes in gastric cancer in certain tissue.

### Example 1

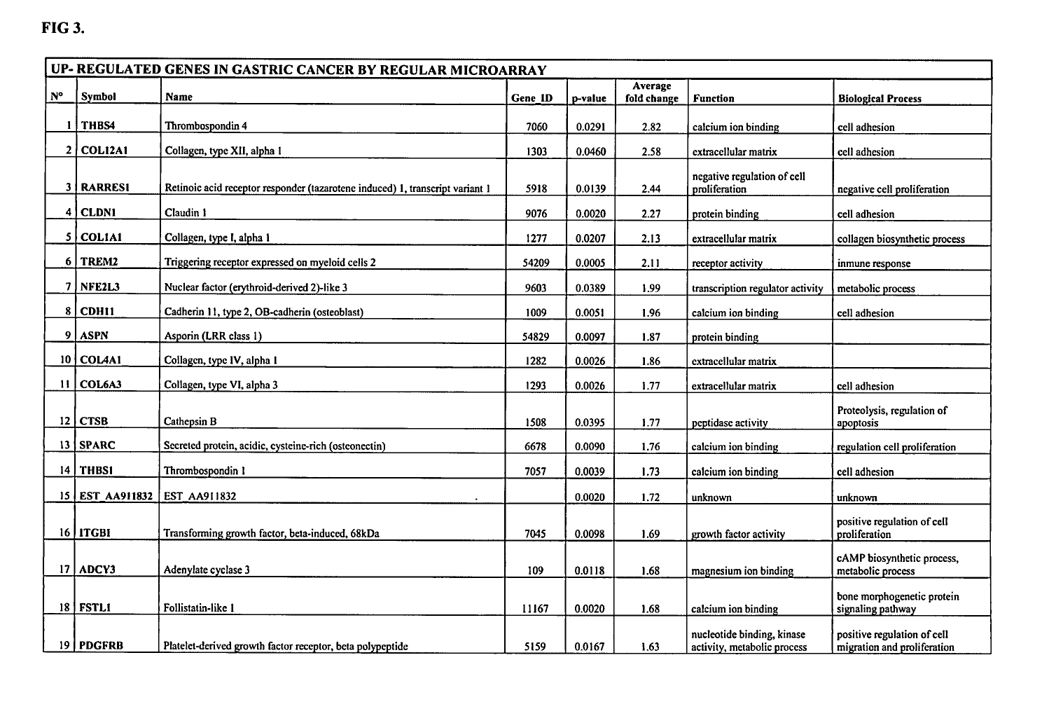

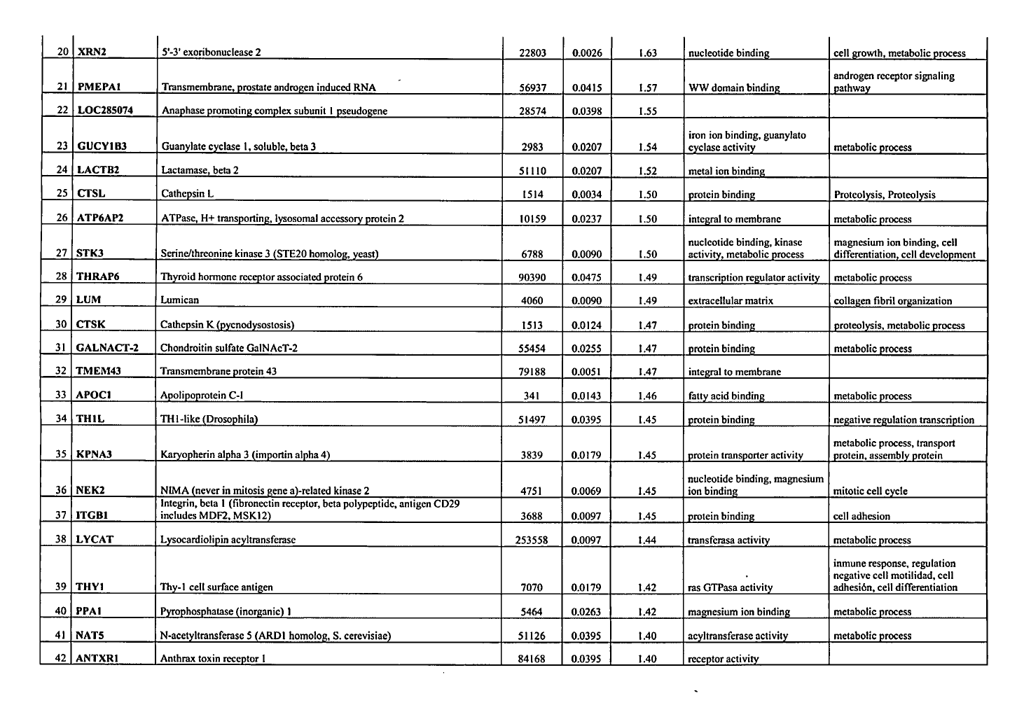

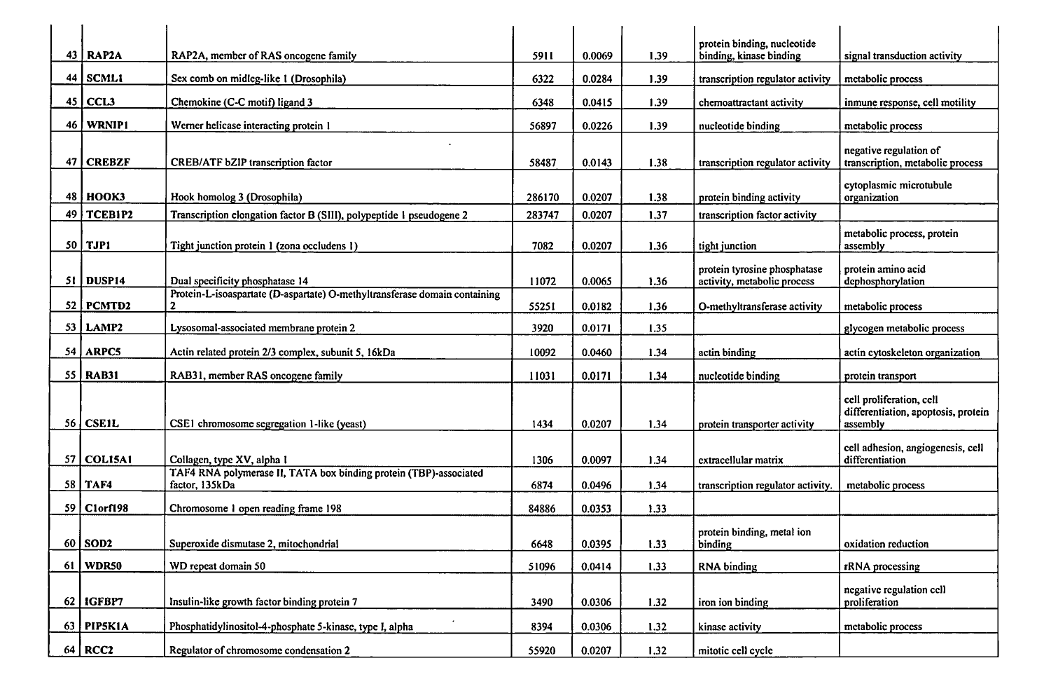

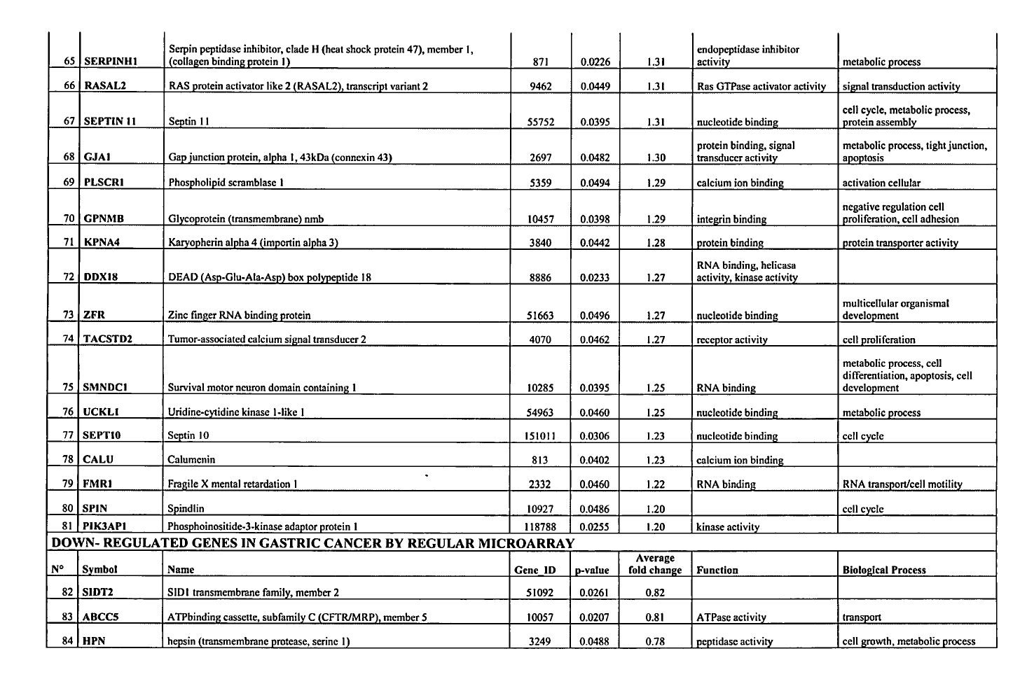

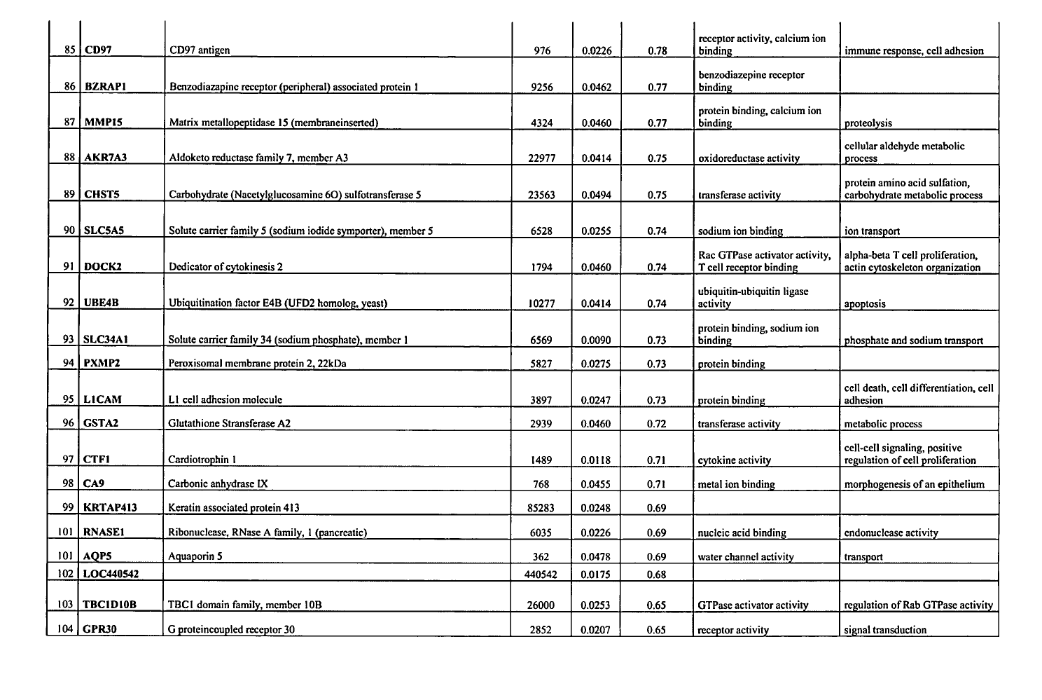

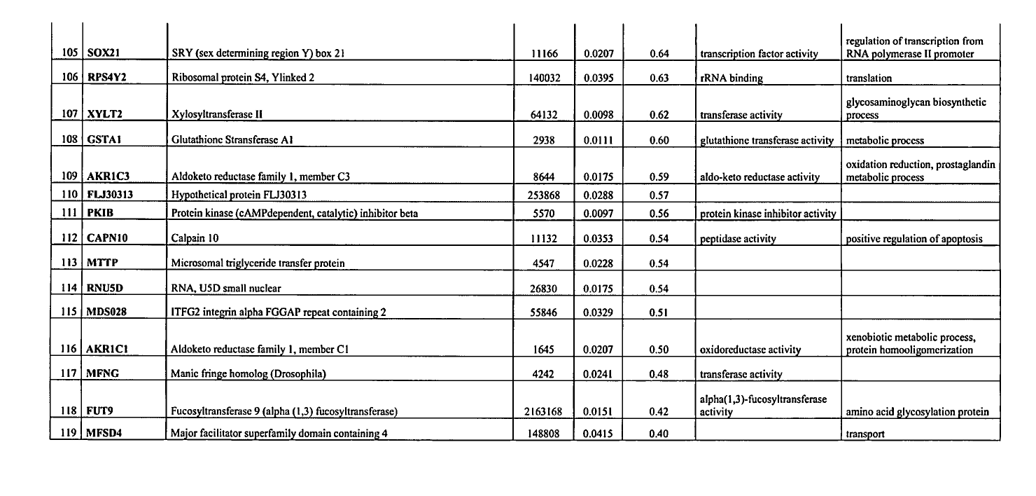

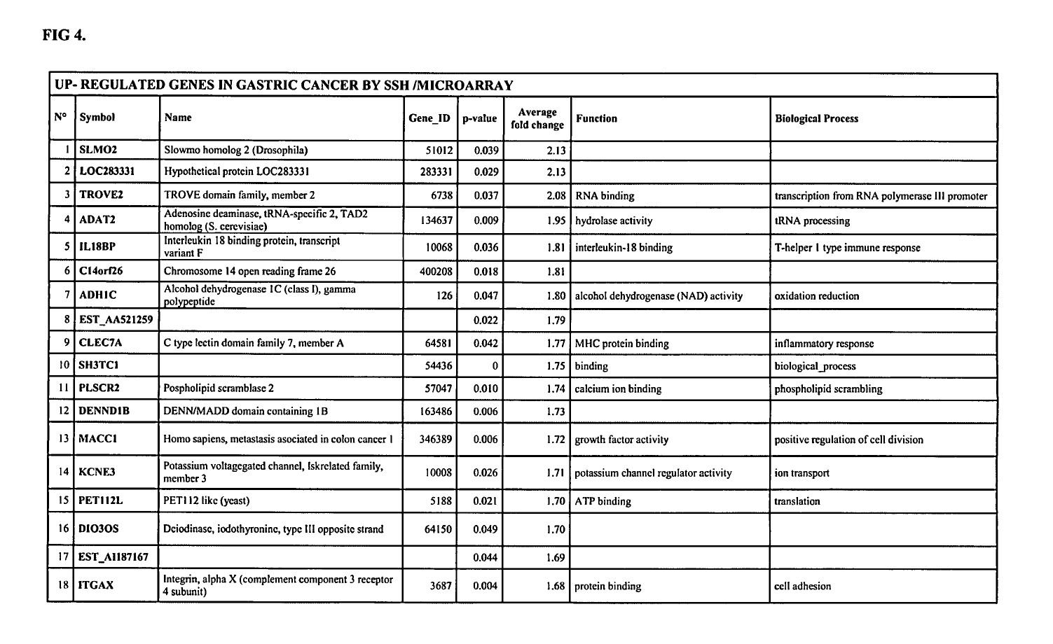

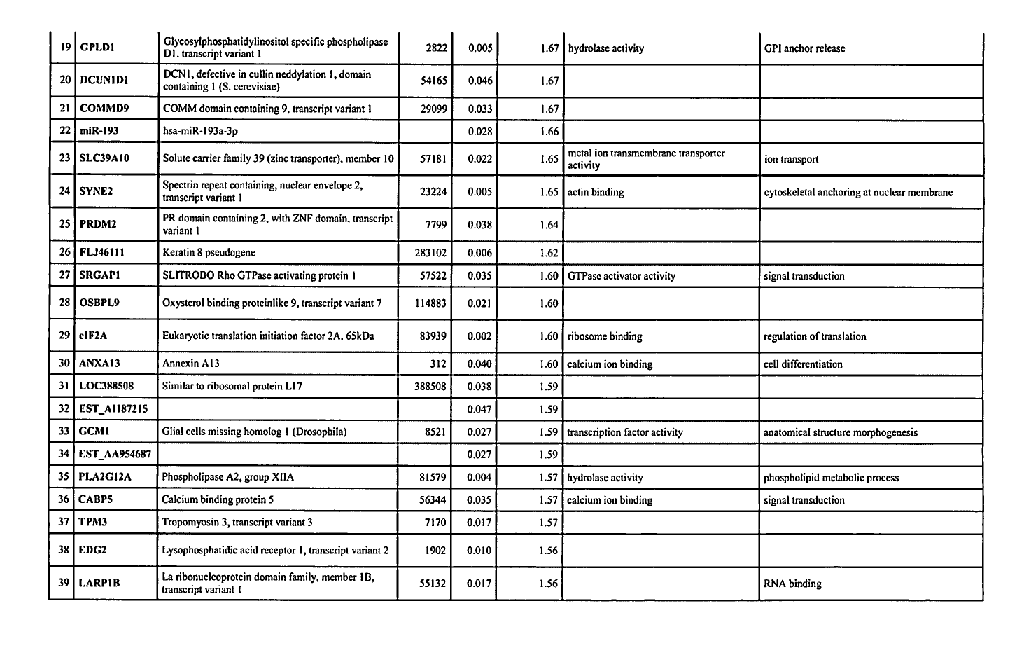

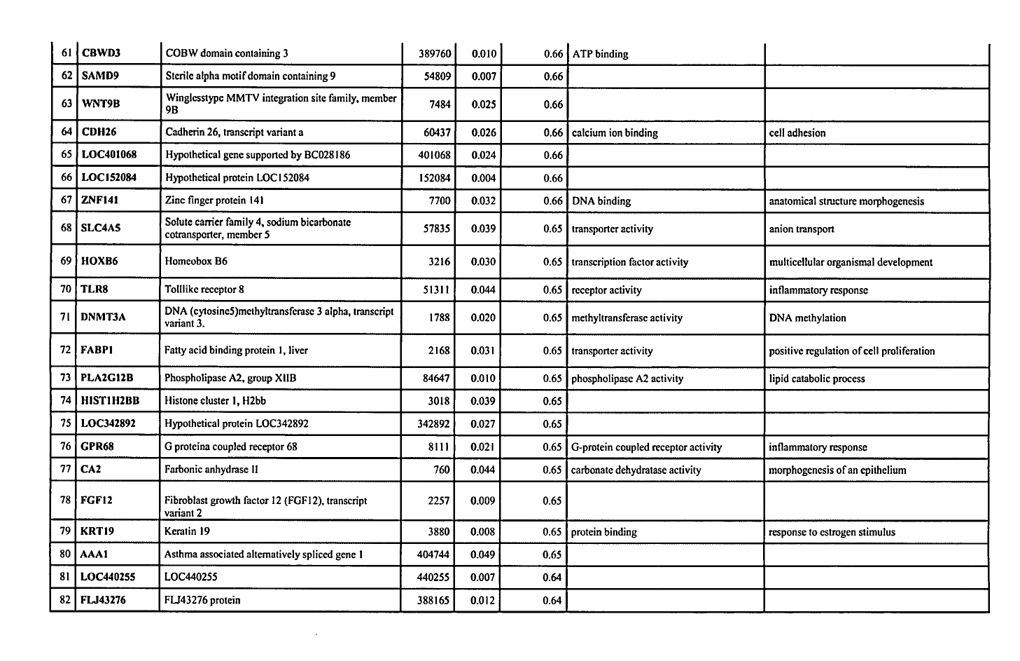

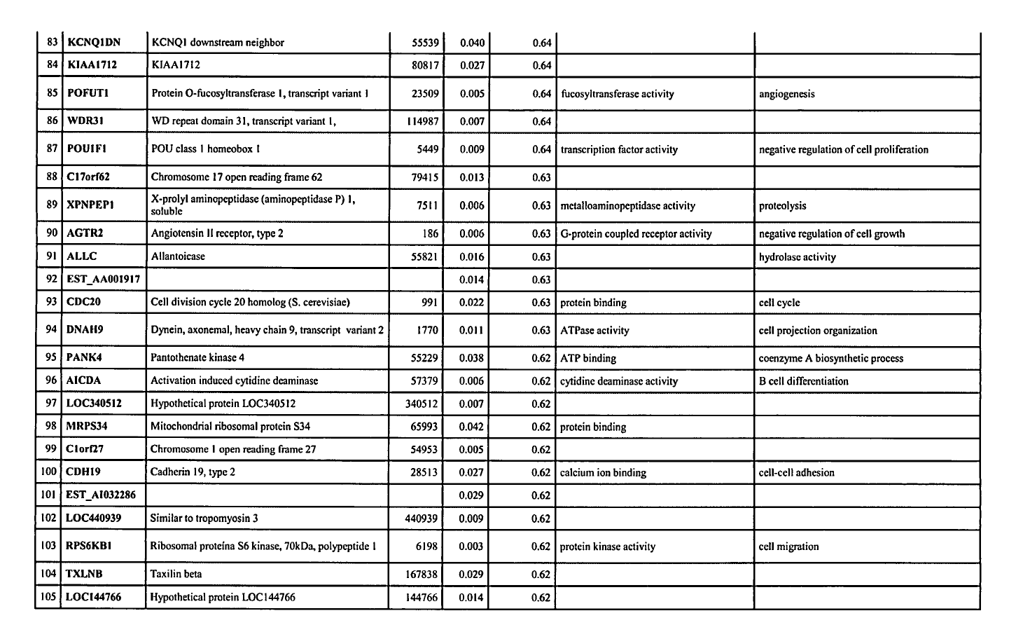

In order to clarify gastric carcinogenesis, a pairing analysis was performed among the 12 samples studied through both strategies. 119 differentially expressed genes were identified from the analysis of the regular microarray strategy. 81 corresponded to up-regulated genes and 38 to down-regulated genes. In the SSH-microarray strategy, a total of 149 differentially expressed genes were identified. Of that number, 59 genes corresponded to up-regulated and 90 genes to down-regulated genes. Both results were statistically significant (p<0.05). FIGS. 3 and 4 show the differential genes obtained from each strategy, including the fold change in the Tumor-Adjacent (T/A) ratio, the statistical significance (p-value) as well as the function and the biological process each gene is involved in.

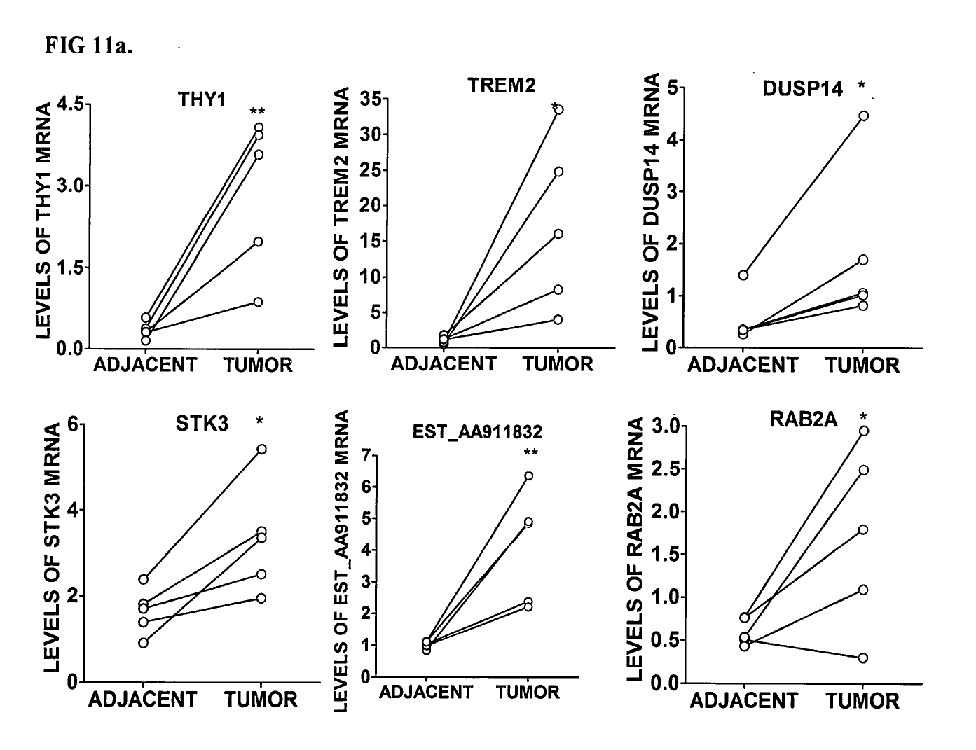

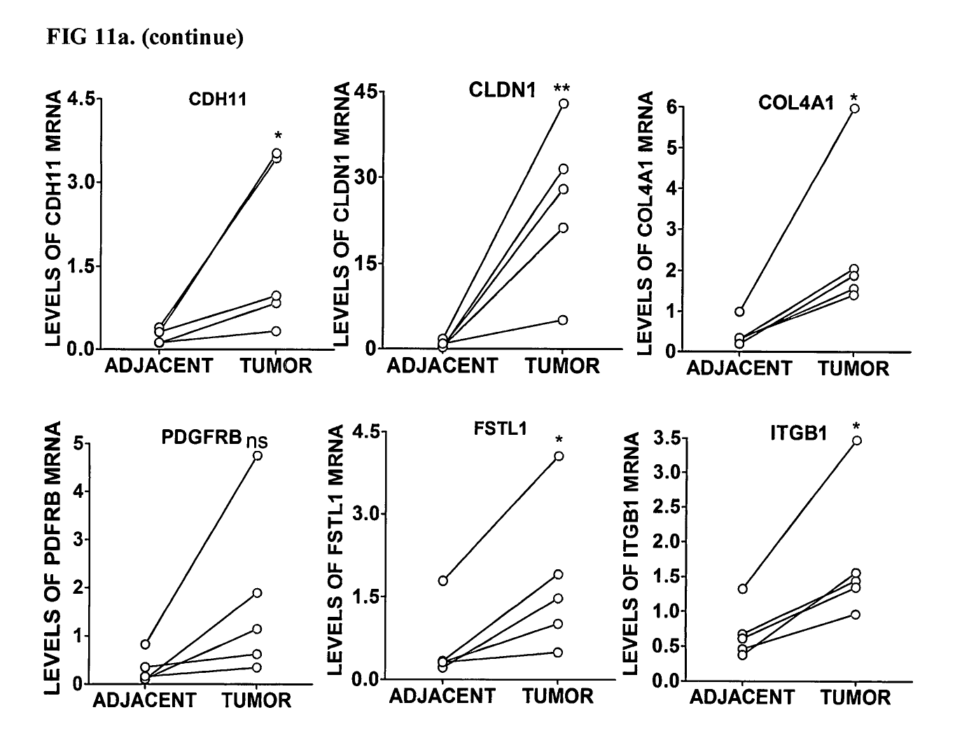

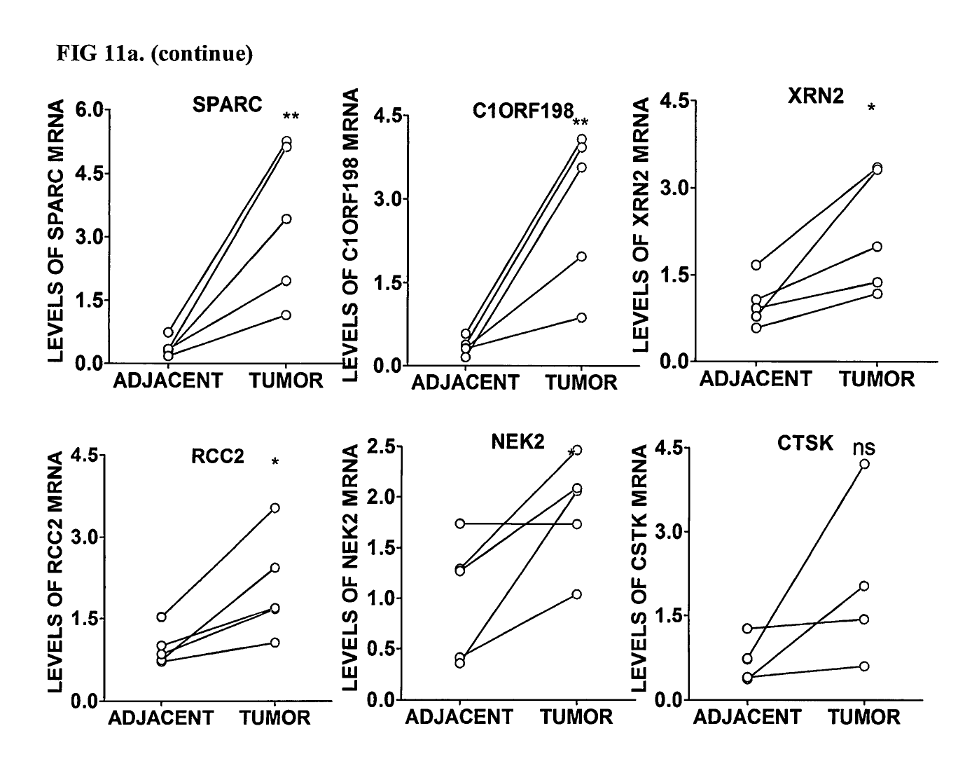

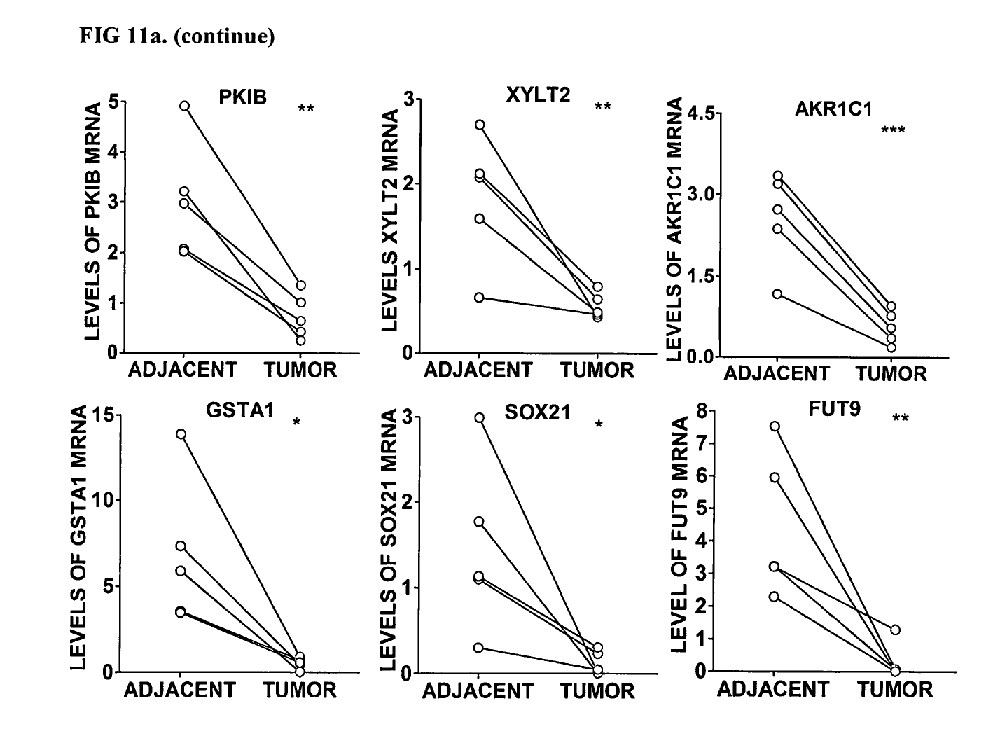

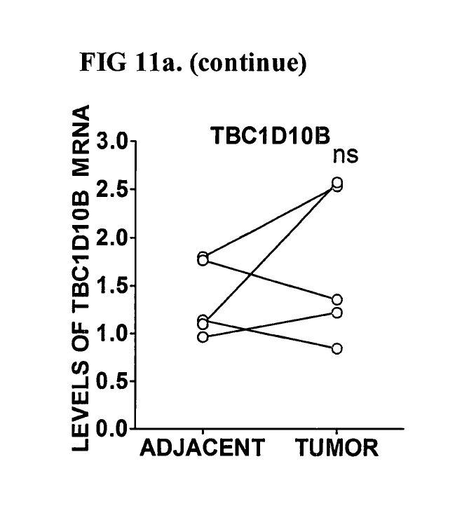

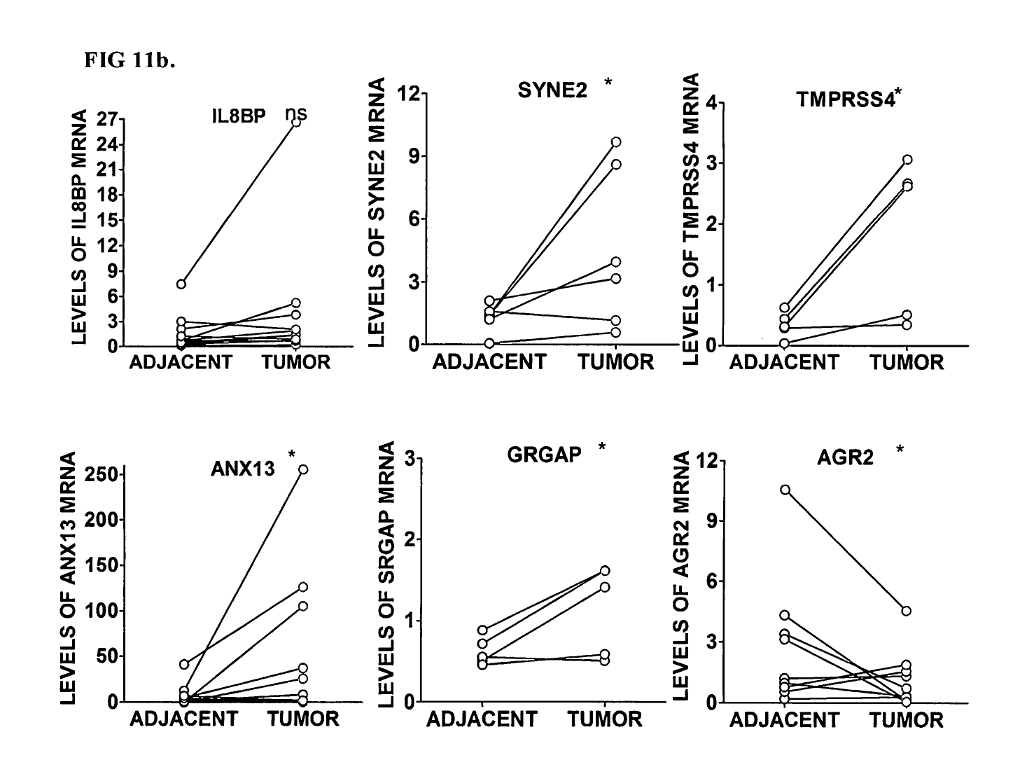

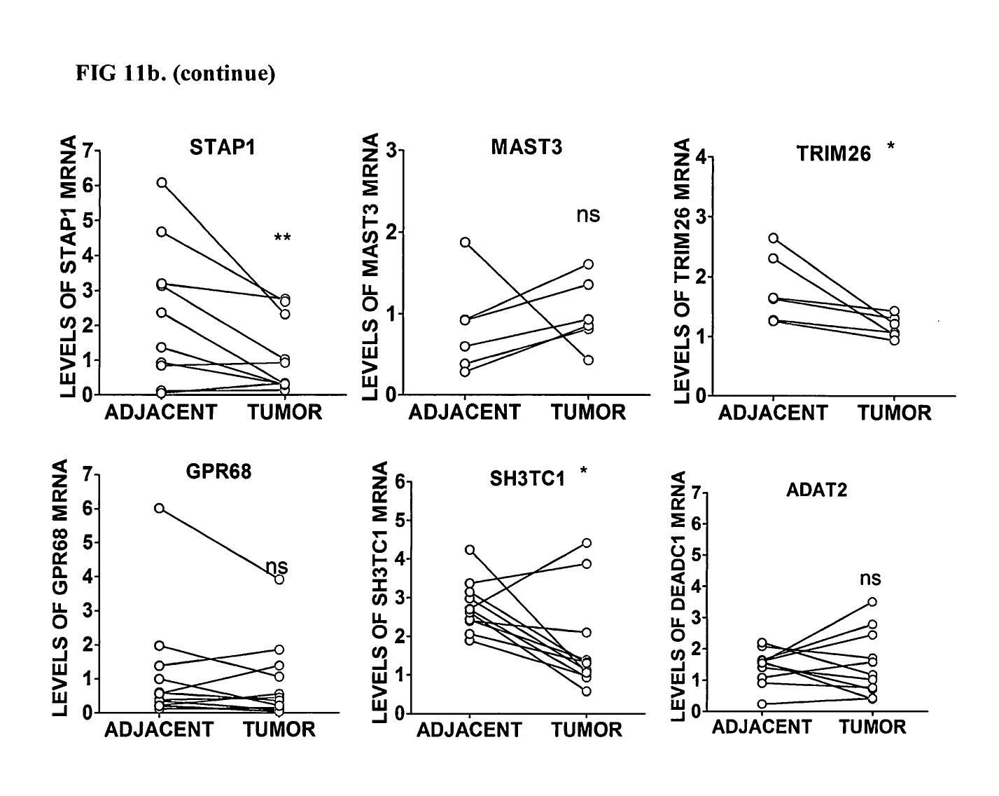

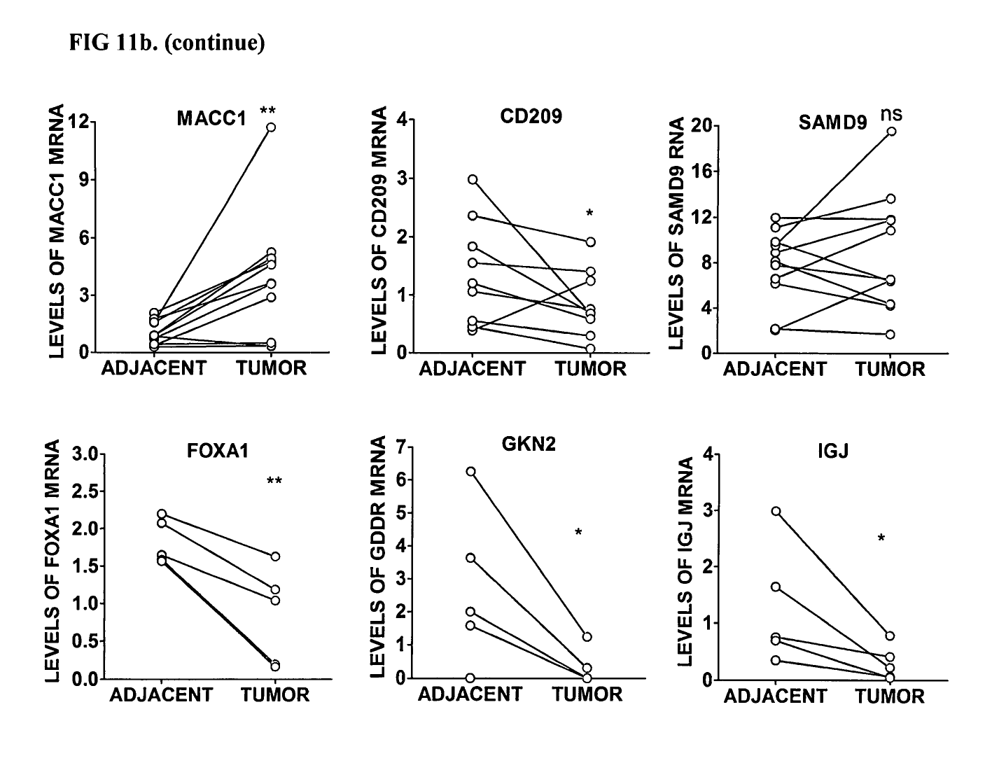

In order to validate the information obtained from the regular microarray strategy, 25 selected genes were validated by quantitative real-time PCR. 19 corresponded to up-regulated genes (THY1, TREM2, CTSK, STK3, DUSP14, CDH11, EST_AA911832, RAB2A, CLDN1, PDGFRB, ITGFBI, COL4A1, NEK2, XRN2, c1orf138, FSTL1, SPARC, RCC2, TBC1D10B) and 6 to down-regulated genes (AKR1C1, GSTA1, SOX21, FUT9, XYLT2, PKIB). The real-time analysis confirmed the differential expression in 23 genes with a statistical significance; p<0.05. Two genes followed the expression trend (CTSK and PDGFRB), but the difference did not attain statistical significance and only one gene (TBC1D10B) exhibited data that contradict the microarray result. Thus, real time-PCR confirmed the data in 96% of the cases (FIG. 11a).

18 genes were validated by real time PCR in the SSH/microarray strategy. 10 genes corresponded to up-regulated genes (IL8BP, SYNE2, TMPRSS4, ANX13, GRGAP, MAST3, SH3TC1, DEADC1, MACC1, SAMD9) and 8 to down-regulated genes (AGR2, STAP1, TRIM26, GPR68, CD209, FOXA1, GDDR, IGJ). Data was confirmed in 12 out of the 18 genes (with a statistical significance; p<0.05). Two genes followed the expression trend (IL18P and GPR68), but the difference was not statistically significant and 4 genes (DEADC1, SH3TC1, MAST3 and SAMD9) showed opposite results. In conclusion, 66.7% of selected genes were validated (FIG. 11b).

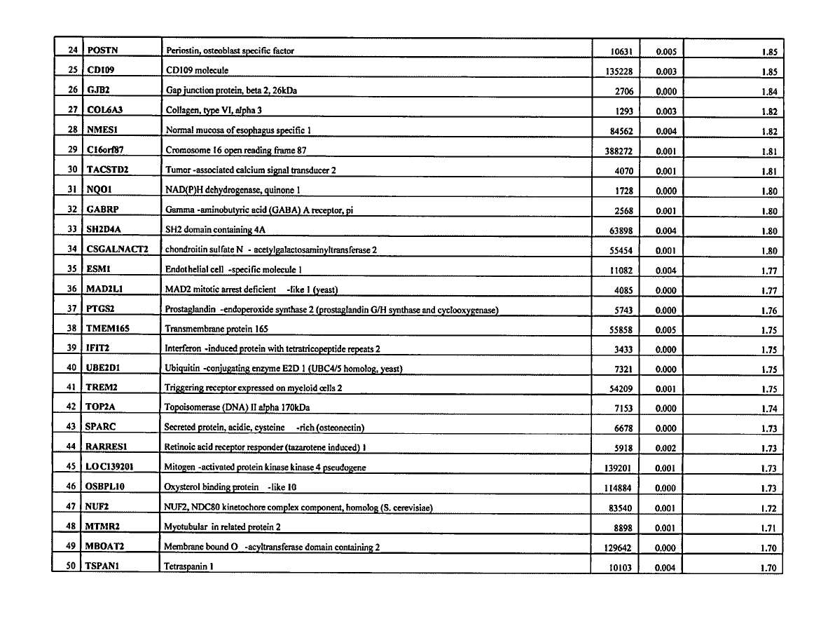

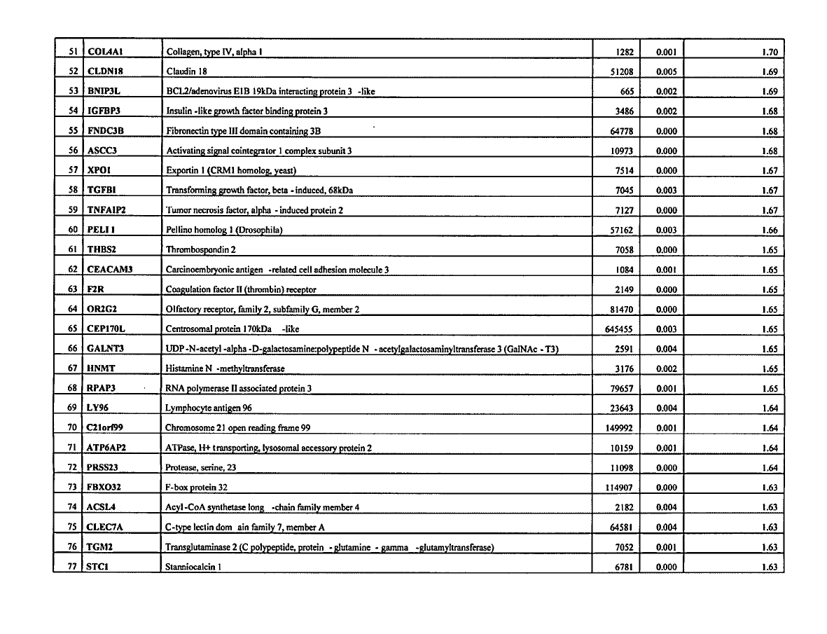

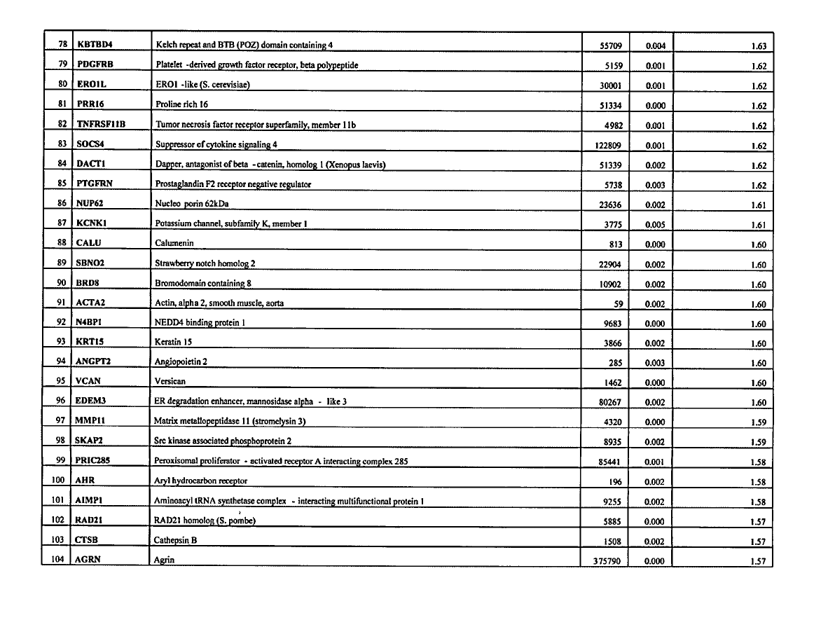

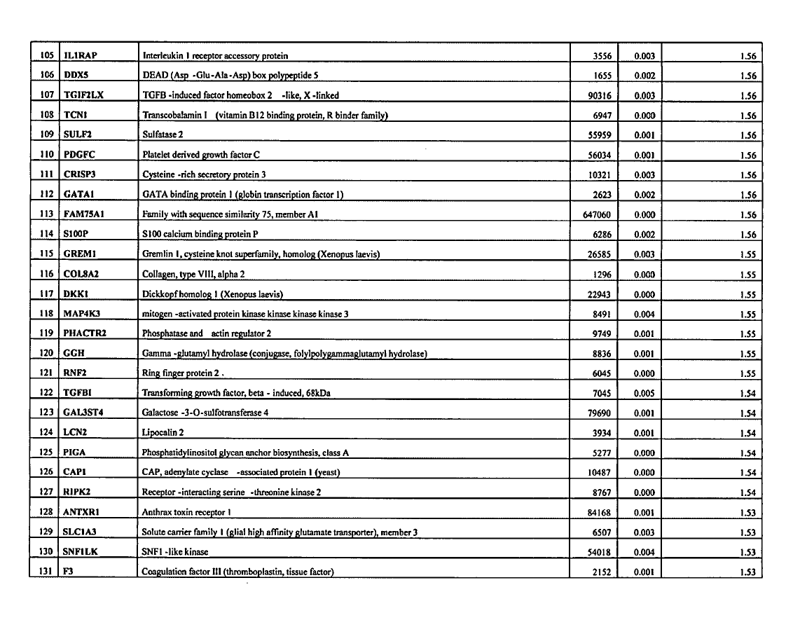

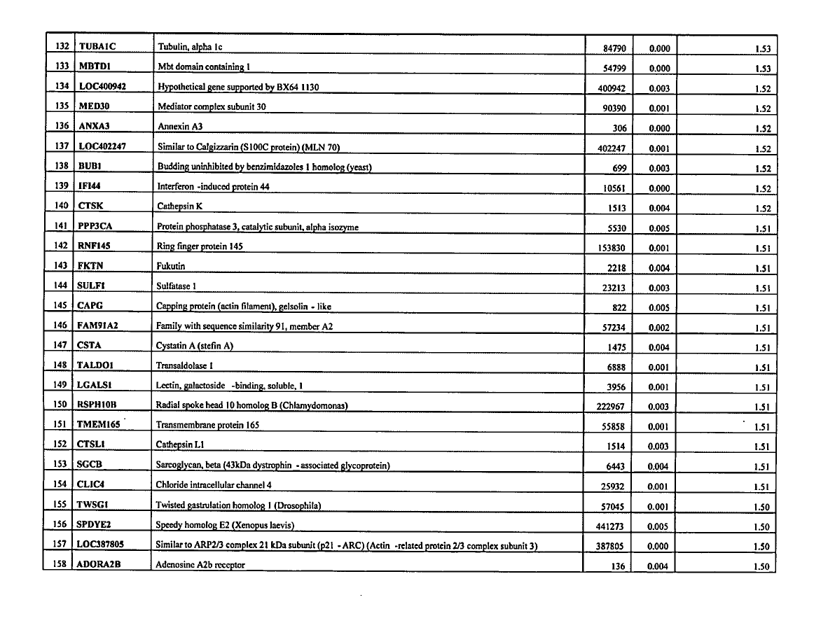

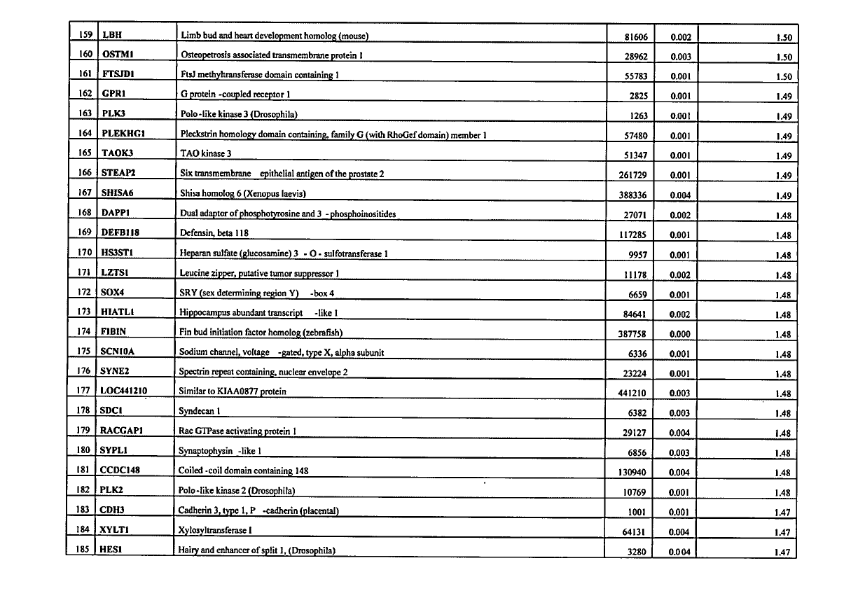

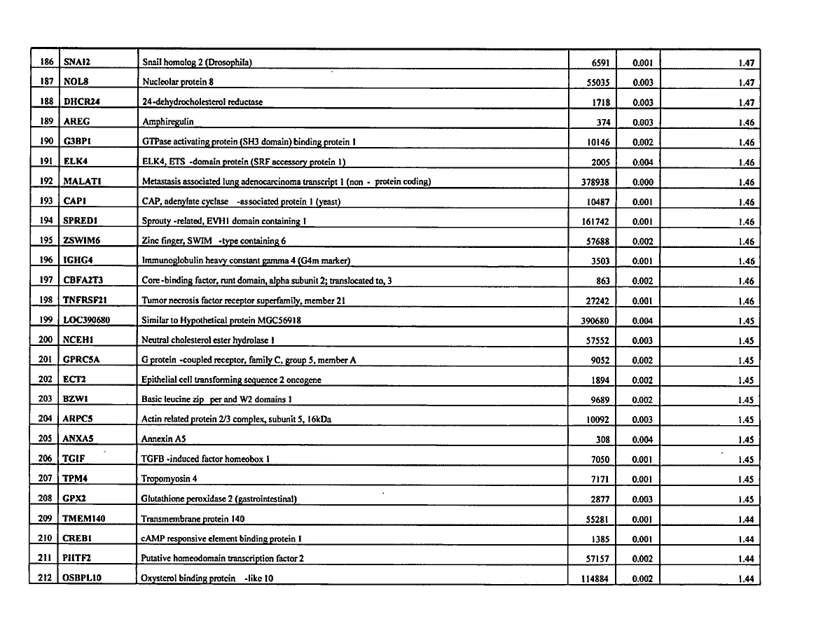

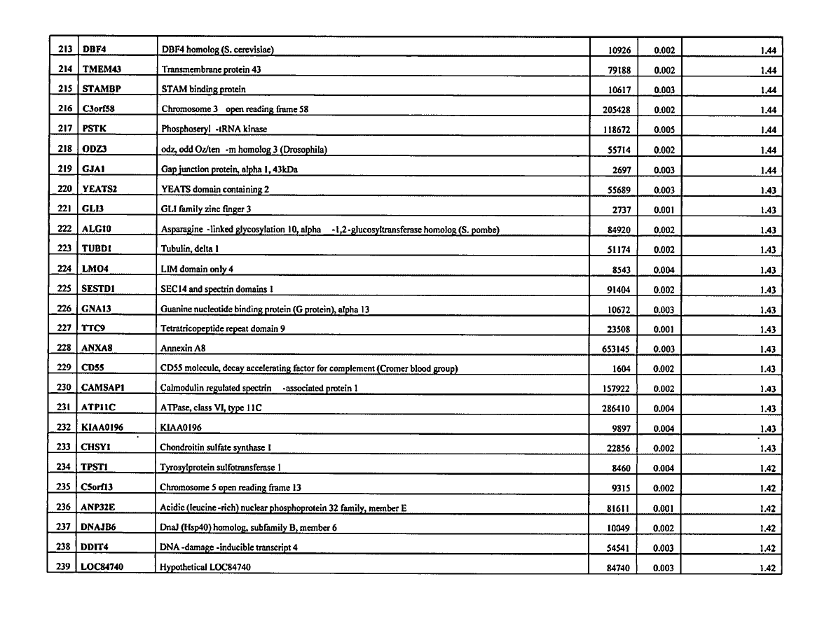

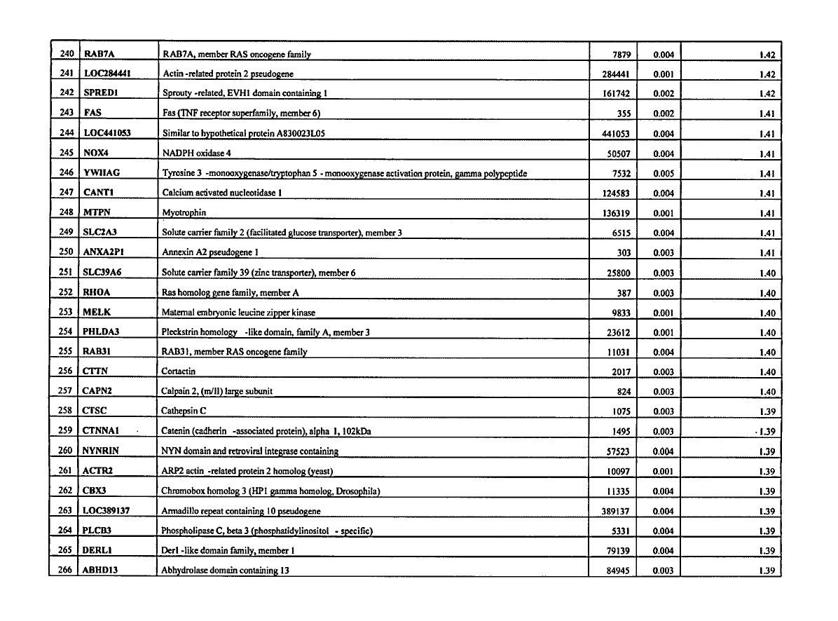

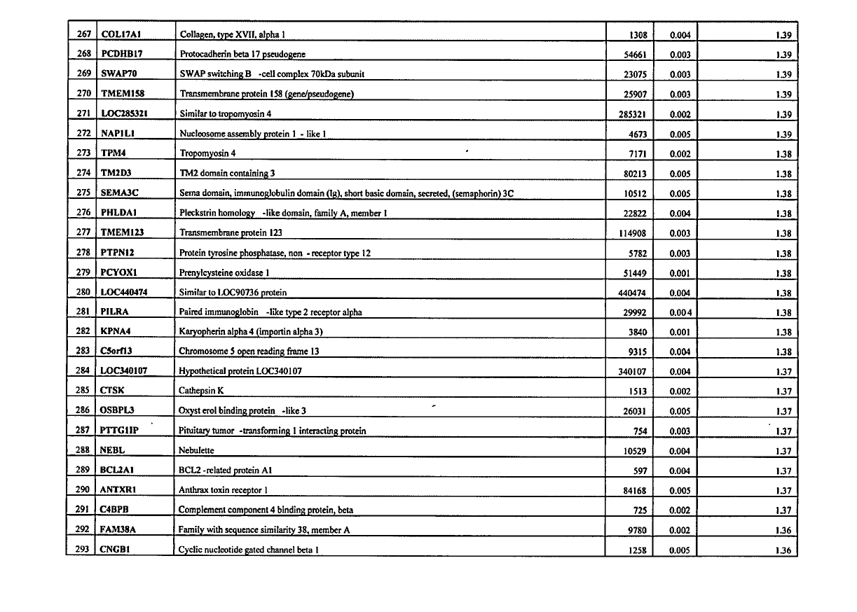

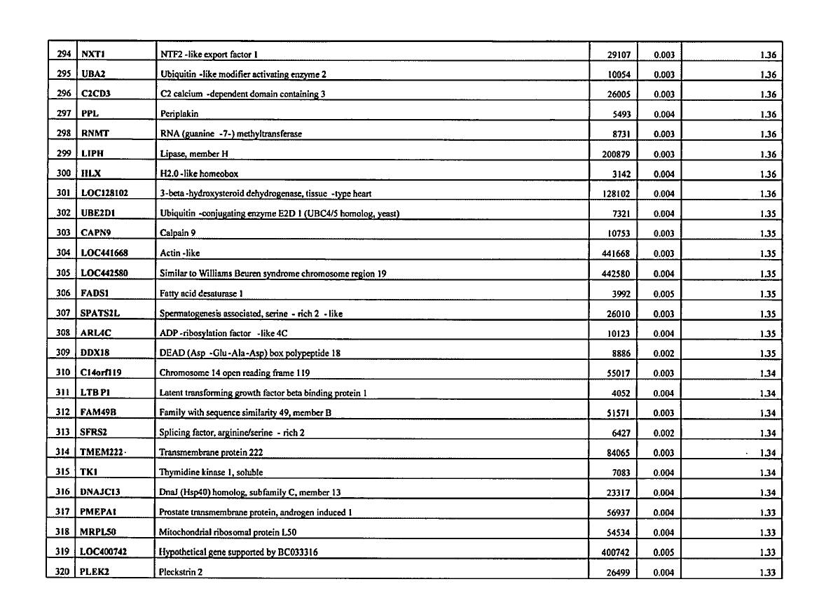

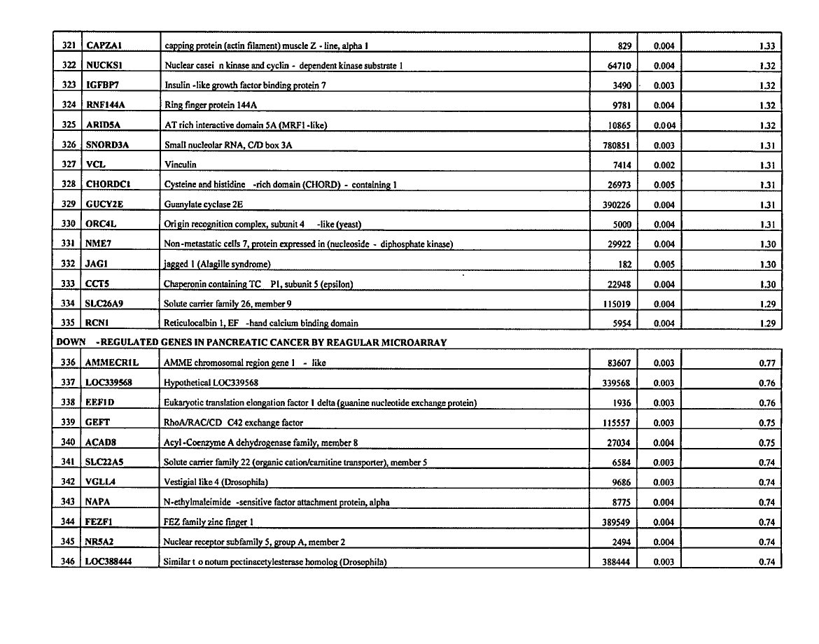

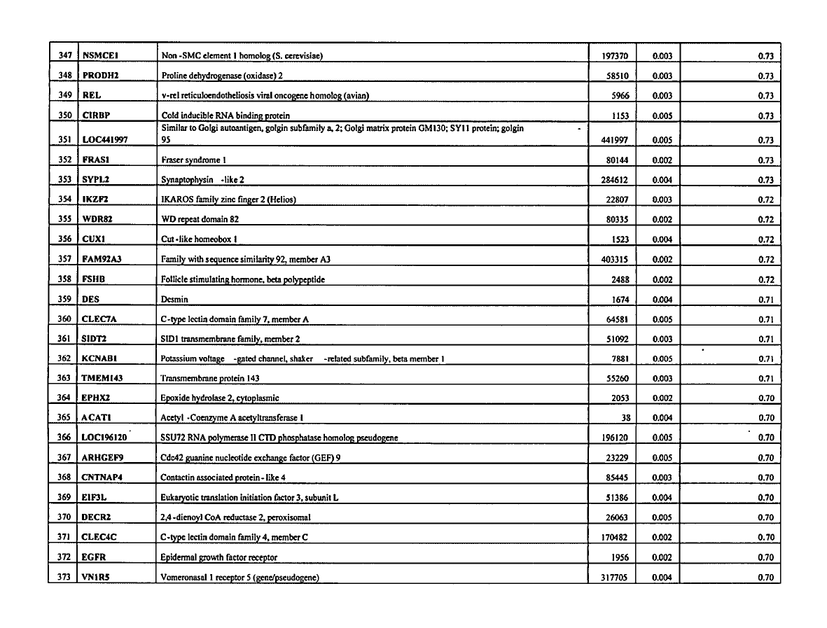

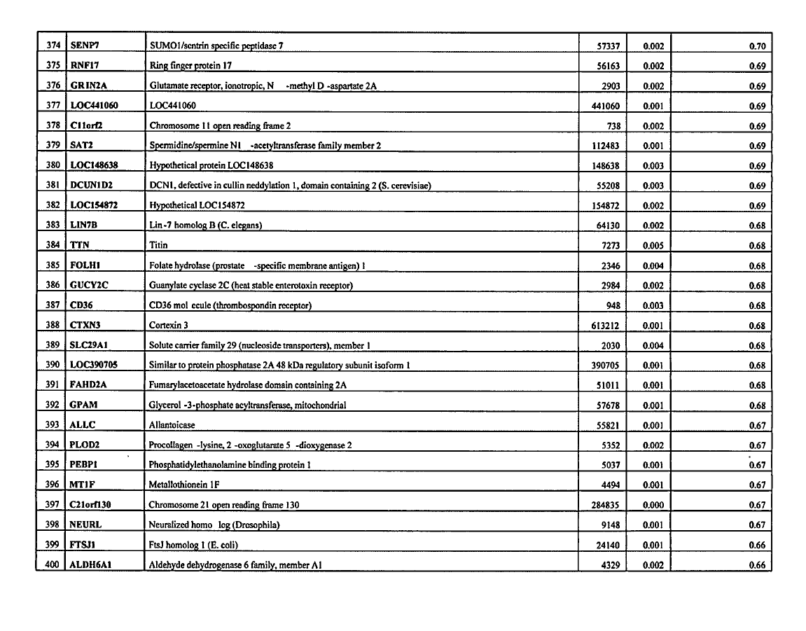

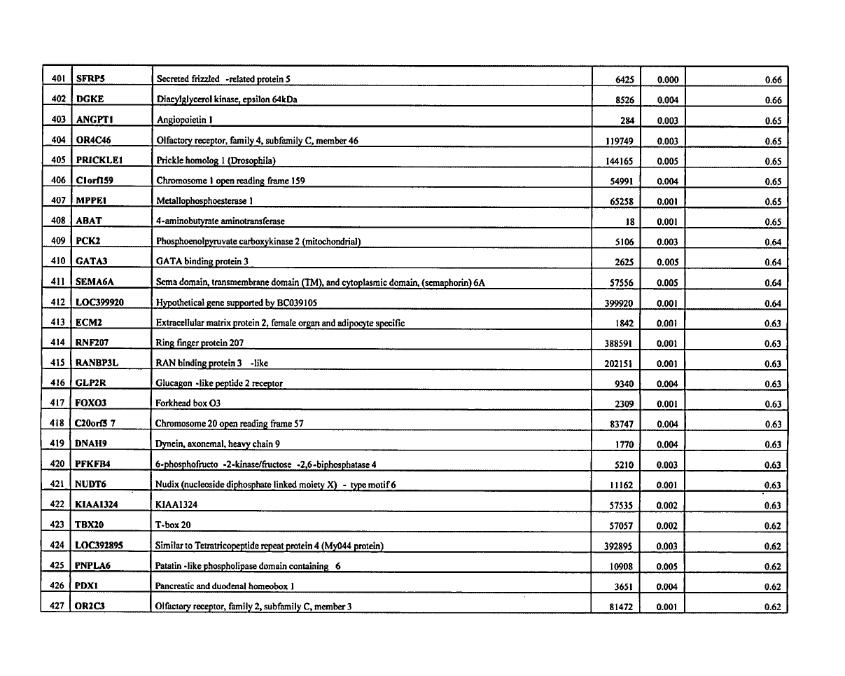

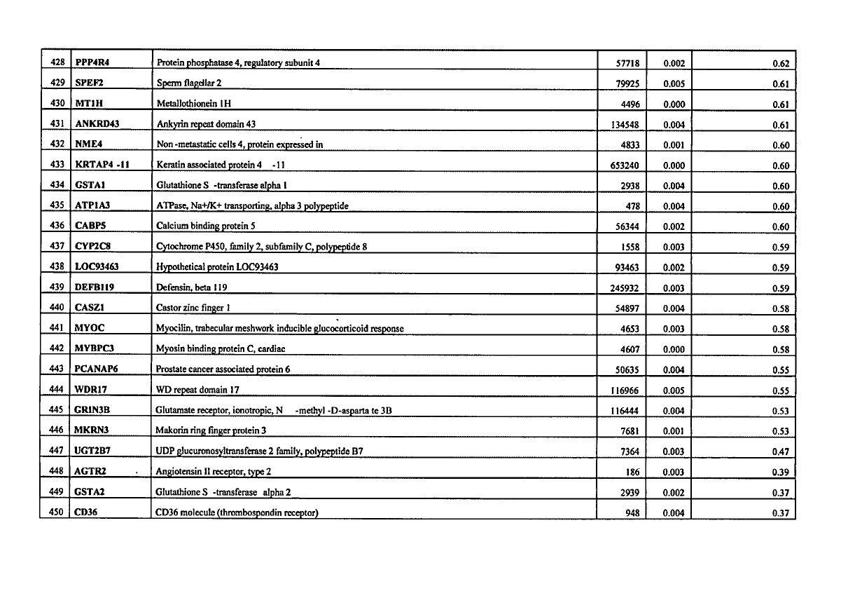

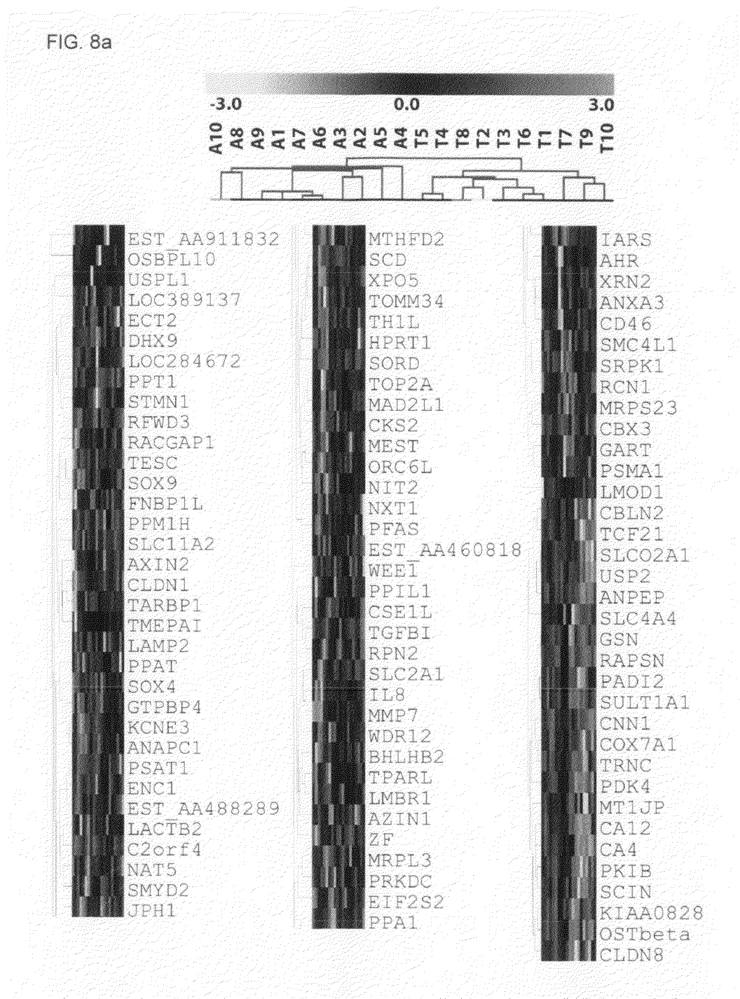

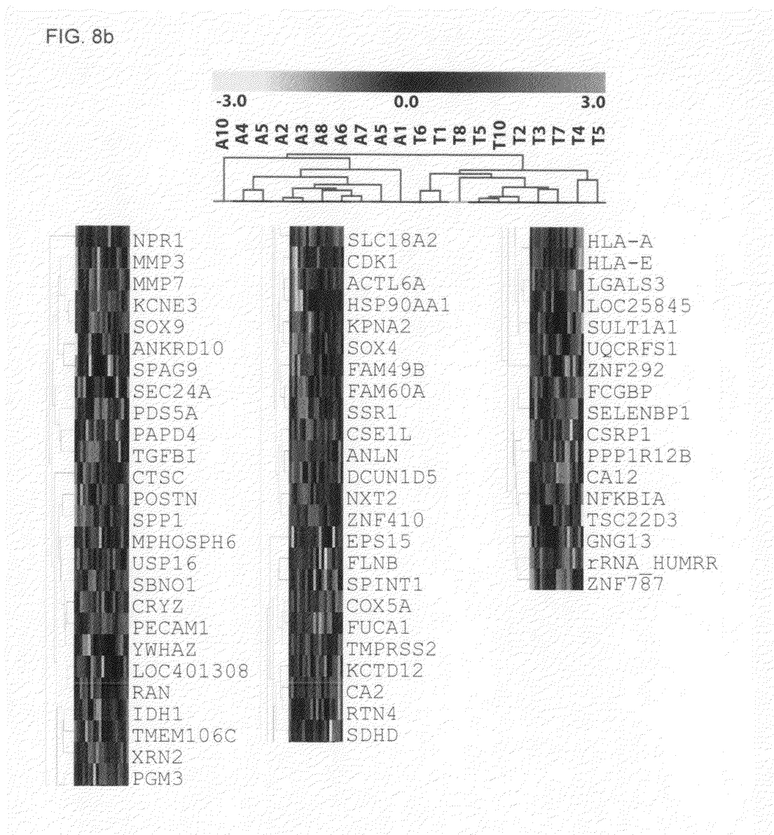

When an unsupervised hierarchical clustering was performed with the normalized “M” values (M=Log₂ red channel intensity/green channel intensity) of each differential gene in each of the cases studied, the genes obtained from both strategies were able of segregate the gastric samples into two large groups: group A, which corresponds to the gastric cancer samples, and group B, which corresponds to the non-cancerous tumor-adjacent tissue (FIGS. 8a and 8b).

It is of note that 53 genes from the regular microarray strategy list were not previously related to gastric carcinogenesis. Among these new genes are the up-regulated genes associated with signal transduction pathways (RAP2A, RASAL2, STK3, ADCY3), cell cycle (RCC2, NEK2, SPIN1, SEPT11, SEPT10), immune response (TREM2), MAP tyrosine kinase (DUSP14), cell proliferation regulation (DDX18), protein from the bone morphogenesis signal pathway (FSTL1), transcription regulation (TAF4, CREBZF, TH1L, THRAP6, ZFR), different metabolic paths (LYCAT, LAMP2, PCMTD2, GUCY1B3, WRNIP1, XRN2, ATP6AP2, PIK3AP1, PPA1, PIP5K1A, ARPC5, GALNACT-2), transfer systems (HOOK3, KPNA3, FMR1), transmembrane receptor activity (ANTXR1, TMEM43), apoptosis (SMNDC1), structural molecular activity (COL12A1) and other genes with an unknown function (c1orf198, LOC285074). Notably, among the new down-regulated genes are genes associated with different metabolic pathways (CHST5, XYLT2, FUT9, MTTP), transfer system (SLC34A1), signal transduction pathways (MDS28), apoptosis (UBE4D, CAPN10), cell proliferation regulation (CTF1, DOCK2), cell organization components (KRTAP4, BZRAP1) and other genes with an unknown function (LOC440542, FLJ30313).

### Example 2

### Differentially Expressed Genes in Colon Cancer

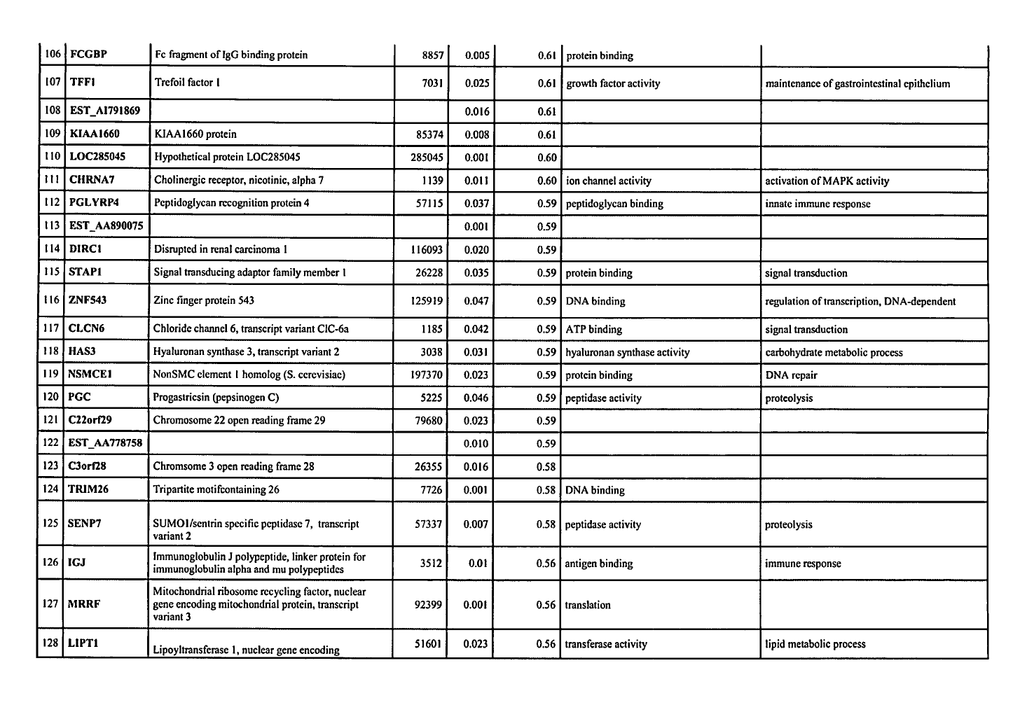

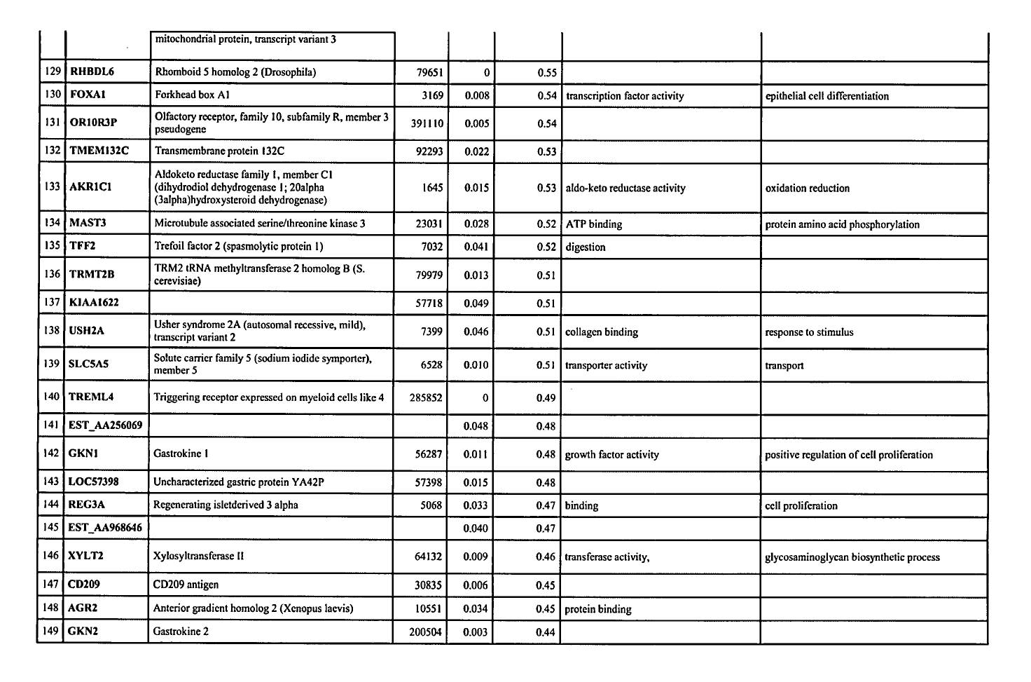

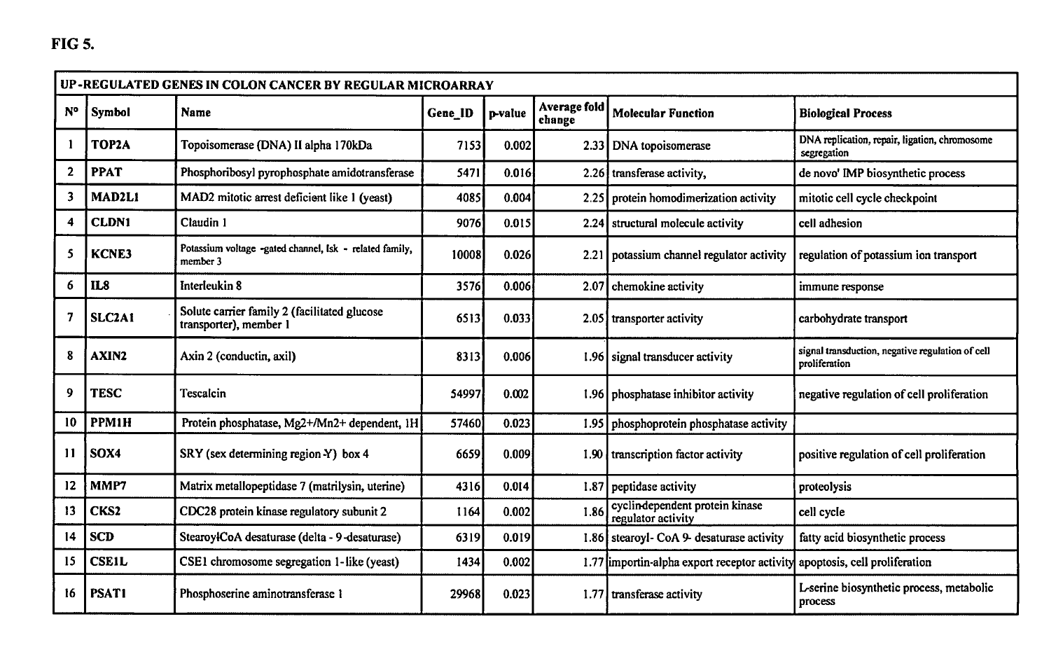

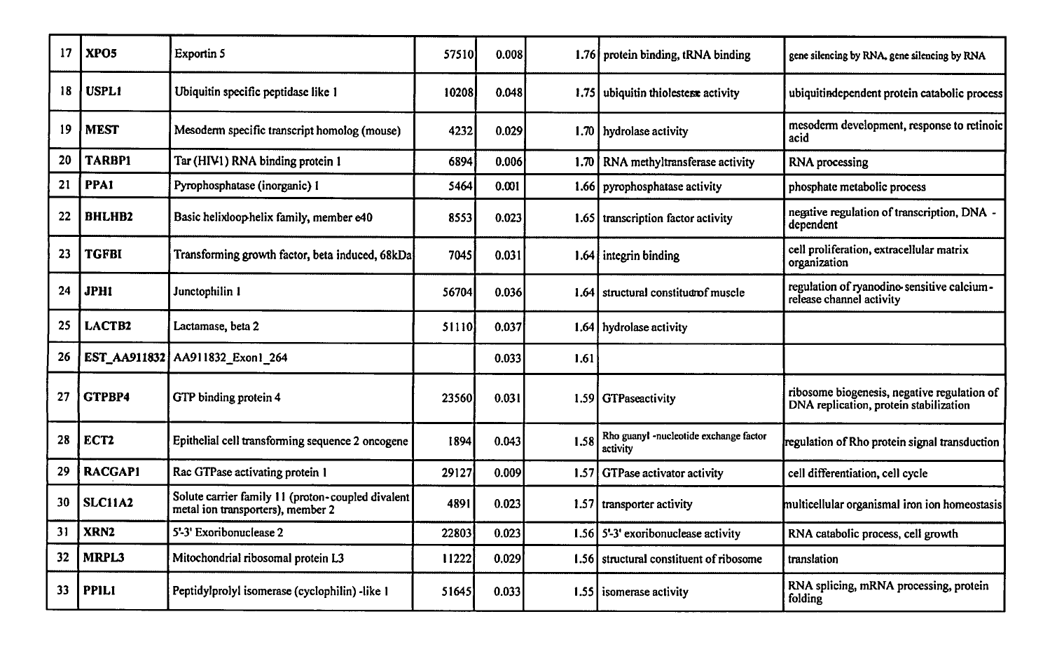

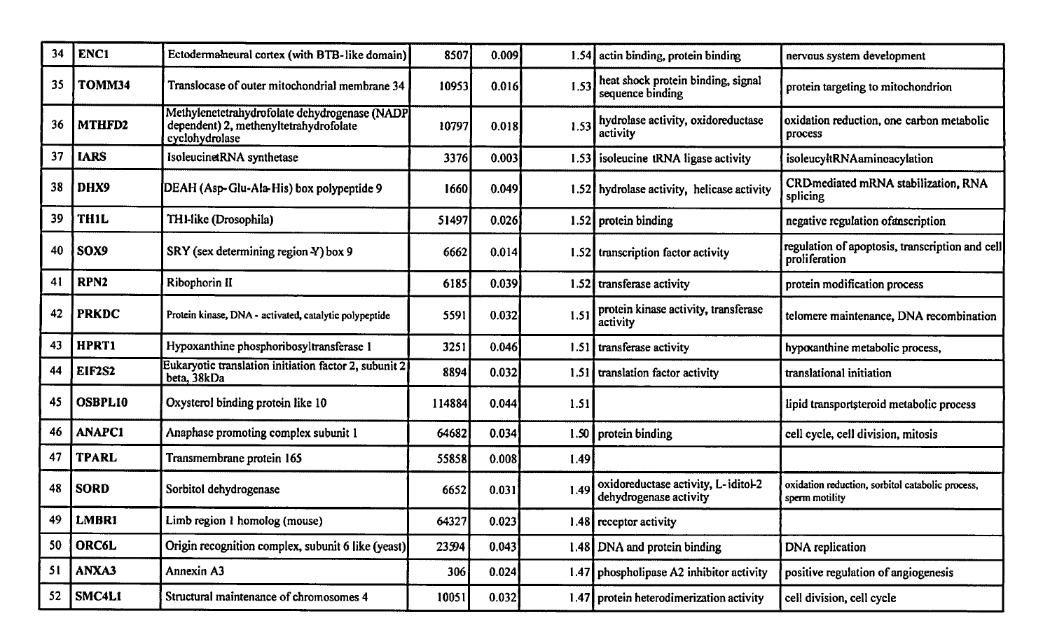

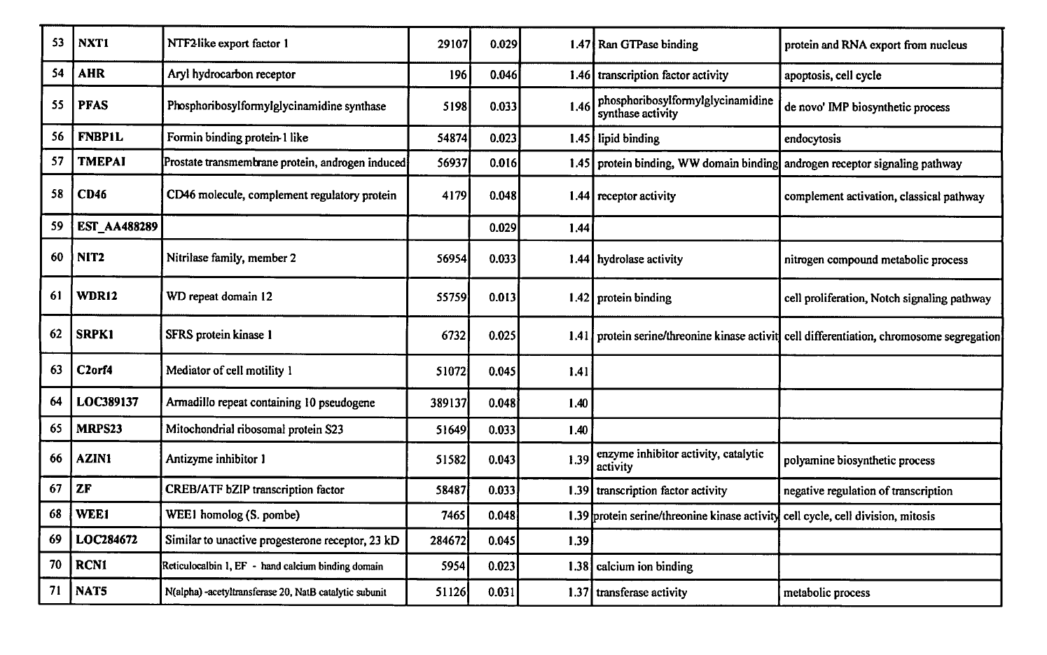

The pairing analysis was made with 10 cases studied through both strategies for the purpose of identifying differentially expressed genes. 103 genes were identified with a differential expression from the direct strategy. 80 of these genes were up-regulated in cancer tissue as compared to their adjacent tissue counterpart, while 23 genes were downregulated in cancer tissue compared to adjacent non-cancerous tissue. 70 differentially expressed genes were identified using the SSH-microarray strategy, 43 genes being up-regulated and 27 down-regulated. The statistical significance of the results of the first analysis corresponds to a p-value below 0.05, while the p-value for the results of the second analysis was below 0.005. The differential genes obtained in each strategy are shown in FIG. 5 and FIG. 6. The fold change values (tumor/adjacent ratio), statistical significance (p-value) and a function and biological process representative of each gene are included.

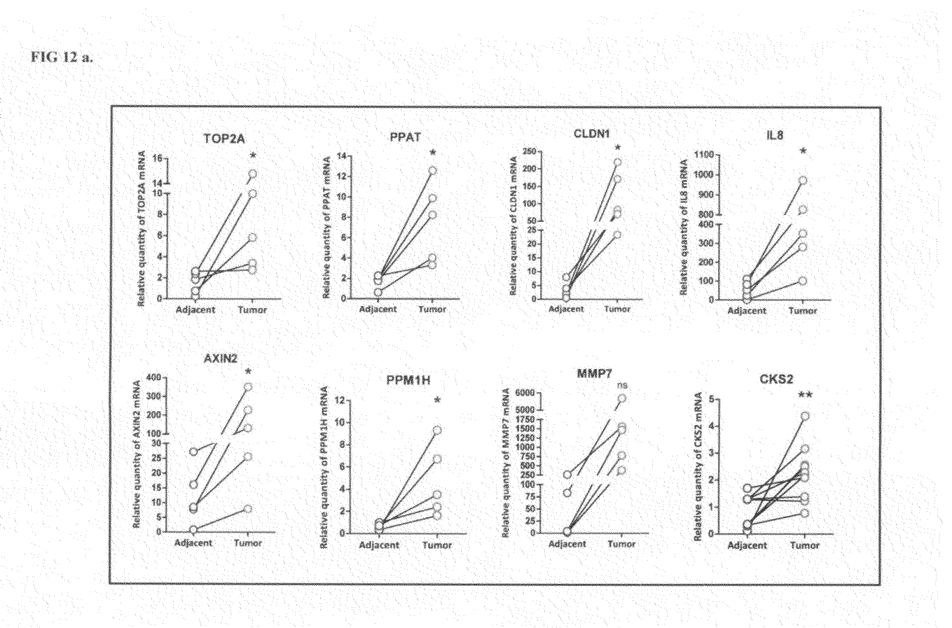

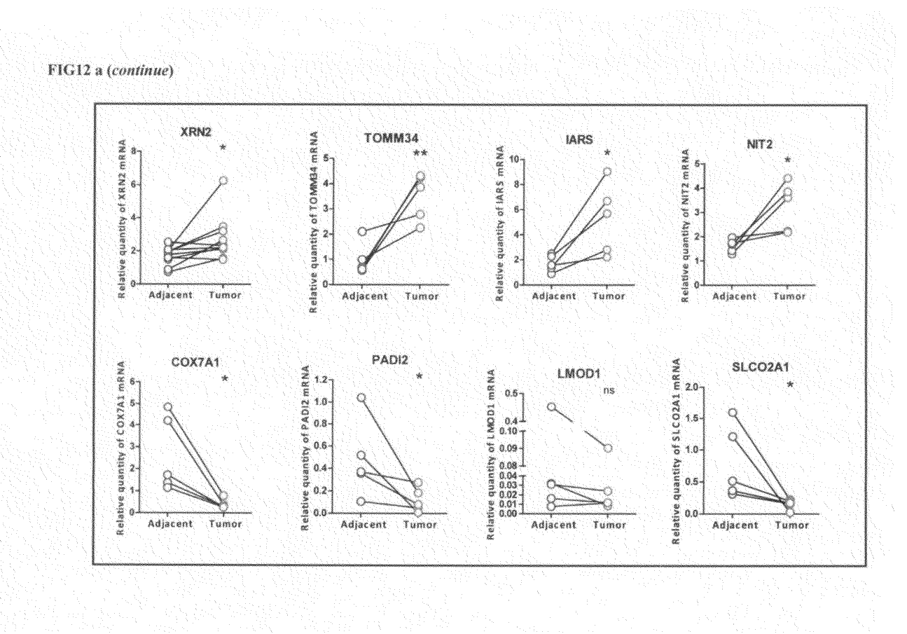

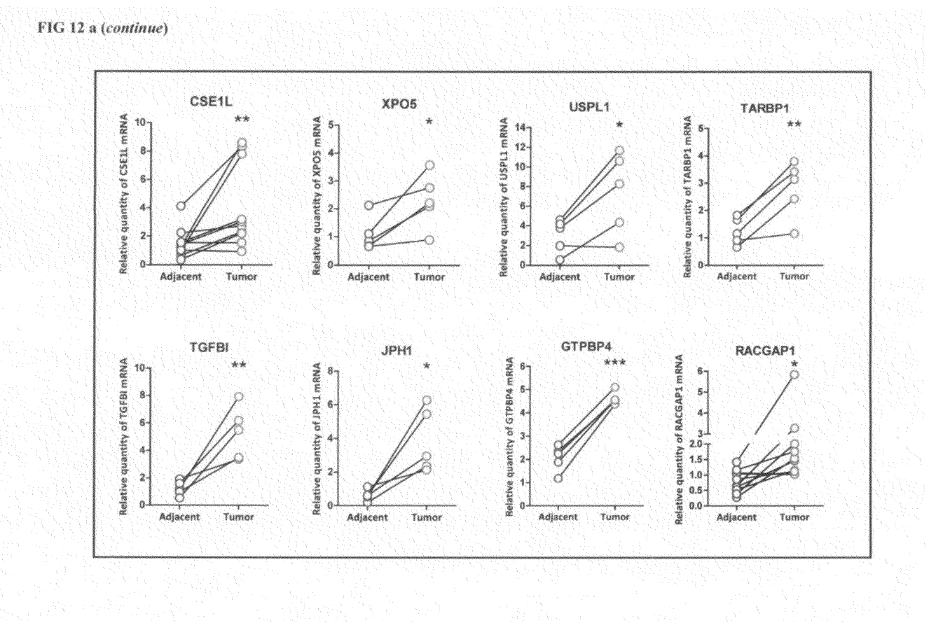

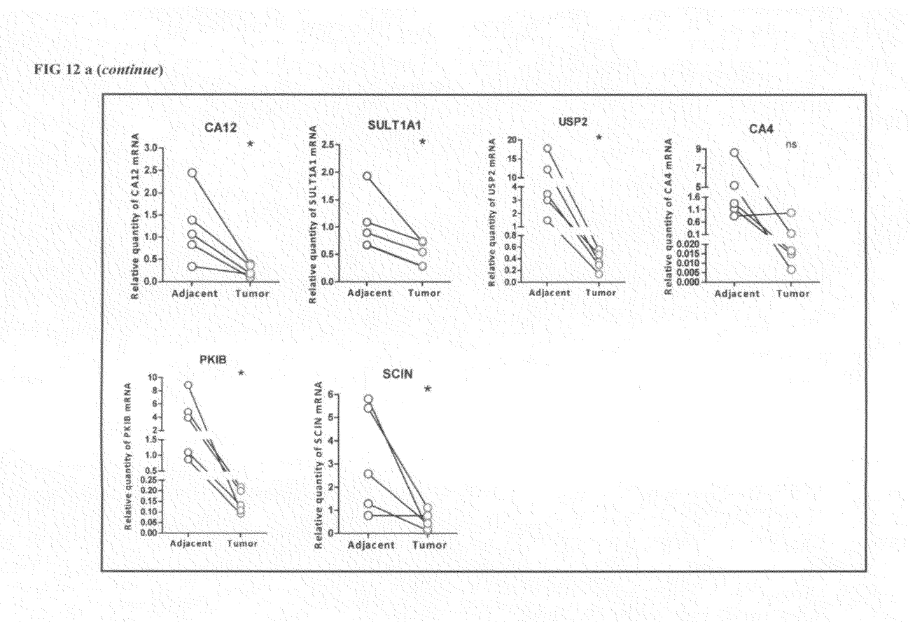

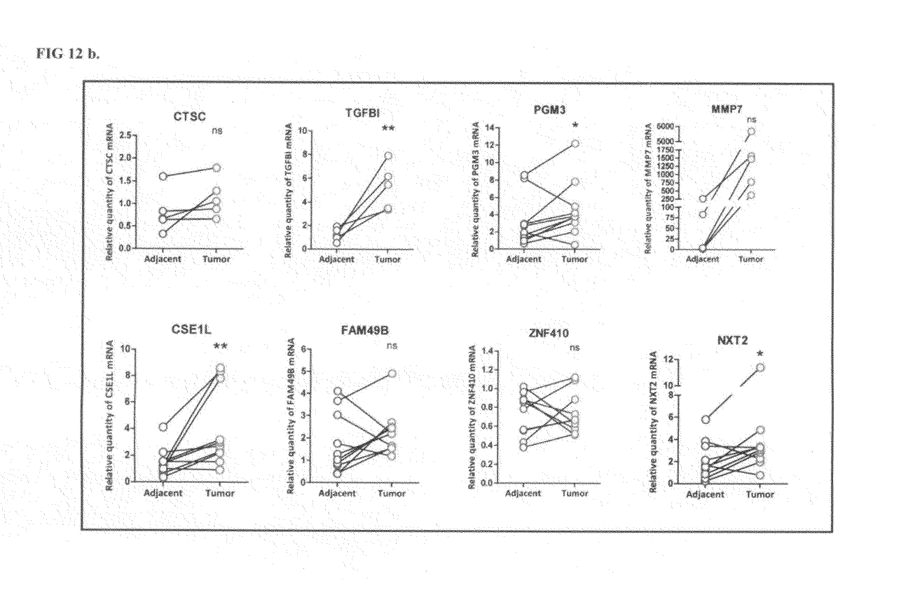

The information obtained from the microarrays was validated. For this purpose, differential genes were selected from each list and real-time PCRs performed. The list of genes to be validated were: TOP2A, PPAT, CLDN1, IL8, AXIN2, PPM1H, MMP7, CKS2, CSE1L, XPO5, USPL1, TARBP1, TGFBI, JPH1, GTPBP4, RACGAP1, XRN2, TOMM34, IARS, NIT2 (up-regulated), and COX7A1, PADI2, LMOD1, SULT1A1, SLCO2A1, CA12, USP2, CA4, PKIB, SCIN (down-regulated). In the SSH-microarray strategy, the genes assessed by RT-qPCR were: CTSC, TGFBI, PGM3, MMP7, CSE1L, FAM49B, ZNF410, NXT2, XRN2, KPNA2 (up-regulated) and FLNB, SULT1A1, NFKBIA, CA12, HLA-A, ZNF292, FUCA1, SELENBP1, CA2 (down-regulated). The real-time PCR analysis of the Regular strategy confirmed the microarray data in 27 genes (90%) (p<0.05). The 3 remaining genes (MMP7, LMOD1, CA4) (10%) showed the correct trend, i.e. although differences were not statistically significant (FIG. 12a). The real-time PCR analysis of the SSH-microarray strategy confirmed the microarray data for 13 genes (68.4%) (p<0.05). Three of the remaining genes (CTSC, MMP7, FAM49B) (15.8%) exhibiteded the correct trend, i.e. although not statistically significant. Finally, there were no significant differences in 3 genes (ZNF410, HLA-A, ZNF292) (15.8%). The result for ZNF410 is explained in part because it has been reported as a normalizing gene in real-time PCR analysis of colon cancer tissue compared to tumor-adjacent colon tissue (FIG. 12b).

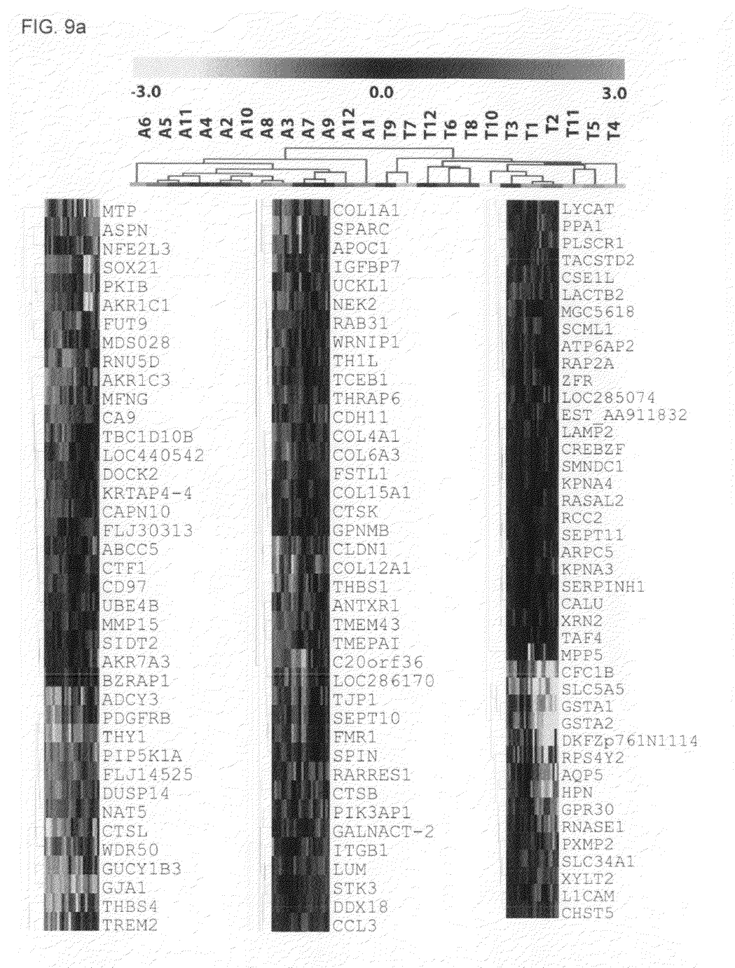

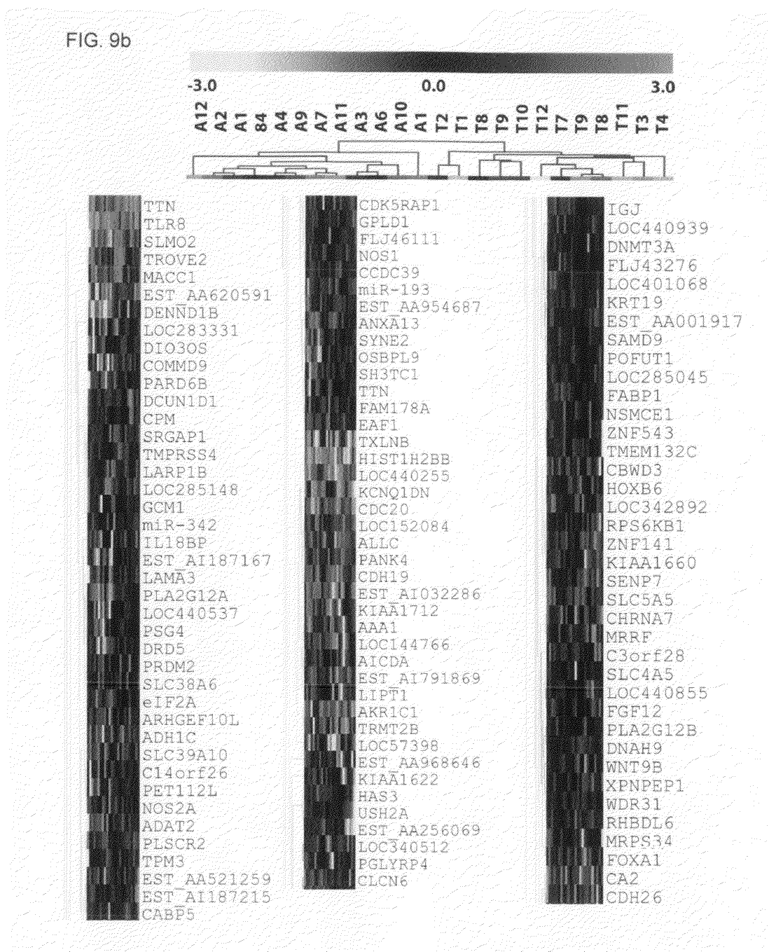

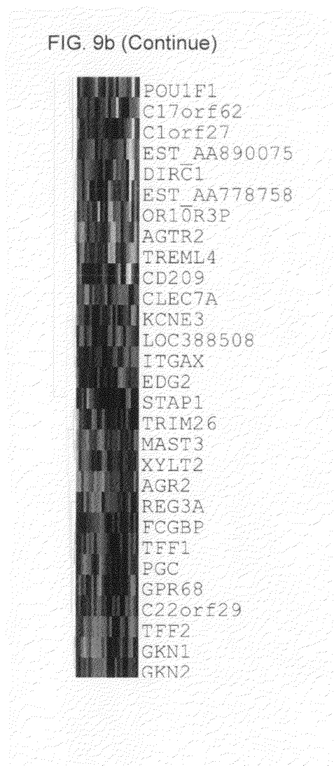

When performing an unsupervised hierarchical clustering with the normalized M values (M=Log 2 intensity channel red/green channel intensity) of each differential gene for each of the cases studied, it appears that the differential genes obtained by both strategies are able to segregate correctly 100% of the cases examined, either 9 cases analyzed with SAM (Regular strategy) or the 10 cases analyzed with LIMMA (SSH/Microarray strategy). There is a clear separation between cancer and adjacent tissues in both cases (FIGS. 9a and 9b).

Remarkably, for 29 genes there is no available information that relates them to colon cancer. Those obtained from the Regular strategy are; C2orf4, CBLN2, COX7A1, EST_AA460818, EST_AA488289, EST_AA911832, FNBP1L, GTPBP4, JPH1, LOC284672, LOC389137, PFAS, RAPSN, RFWD3, TRNC, USPL1 and ZF. While those identified by the SSH/Microarray strategy are; SNORD14B, SLC18A2, EST_AI793123, LOC401308, DCUN1D5, XRN2, ACTL6A, ZNF787, SPINT1, GNG13 and KCTD12. KCNE3 is also included, which was detected with both strategies.

According to the information obtained from PubMed database we found some up-regulated genes that participate or are associated with processes such as transcription regulation (XRN2, ZF, ACTL6A), potassium ionic transmission (KCNE3), negative regulation of DNA replication (GTPBP4), regulation of calcium release activity (JPH1), de novo Inositol Mono Phosphate synthesis (PFAS), endocytosis (FNBP1L), DNA damage checkpoint (RFWD3), death and transport (SLC18A2) and protein catalysis dependent on ubiquitination (USPL1). Finally, we found genes for which no function or participation in any process has been described (C2orf4, EST_AA460818, EST_AA488289, EST_AA911832, LOC284672, LOC389137, DCUN1D5, LOC401308, EST_AI793123, SNORD14B).

We found some down-regulated genes that participate or are associated with processes such as transcription regulation (ZNF787), generation of metabolite precursors and energy (COX7A1), synaptic transmission (RAPSN) morphogenesis (SPINT1), signaling (GNG13), potassium ion transport (KCTD12) and genes for which no function or participation in any process has been described (CBLN2, TRNC).

Eleven genes were identified whose protein product is secreted. Data from the Regular strategy identified IL8 and GSN. While data from the strategy SSH/Microarray identified; POSTN, SAA2, SPP1, MMP3, SPINT1, TMPRSS2 and FCGBP. MMP7 and TGFBI were detected by both strategies (FIGS. 5, 6). Those genes should be considered as potential tumor markers for monitoring disease progression.

### Example 3

### Differentially Expressed Genes in Pancreatic Adenocarcinoma

A pairing analysis was performed on 8 malignant tissues and their respective adjacent non-tumoral tissues in order to identify differentially expressed genes. 450 differentially expressed genes were identified as a result of the analysis of the direct microarray strategy. Of them, 335 corresponded to up-regulated genes and 115 to down-regulated genes with a statistical significance below 0.01%.

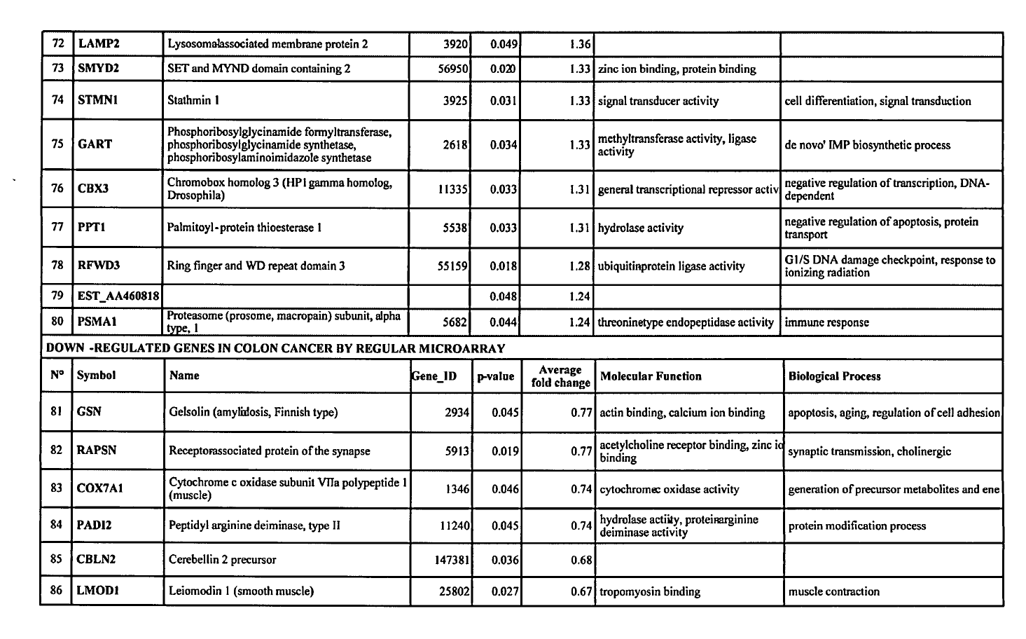

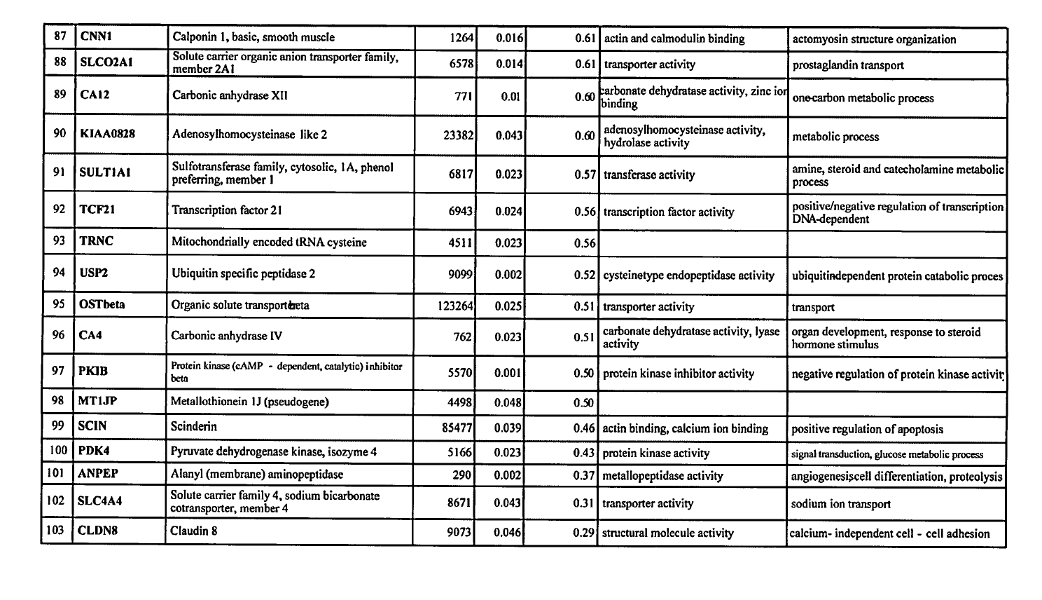

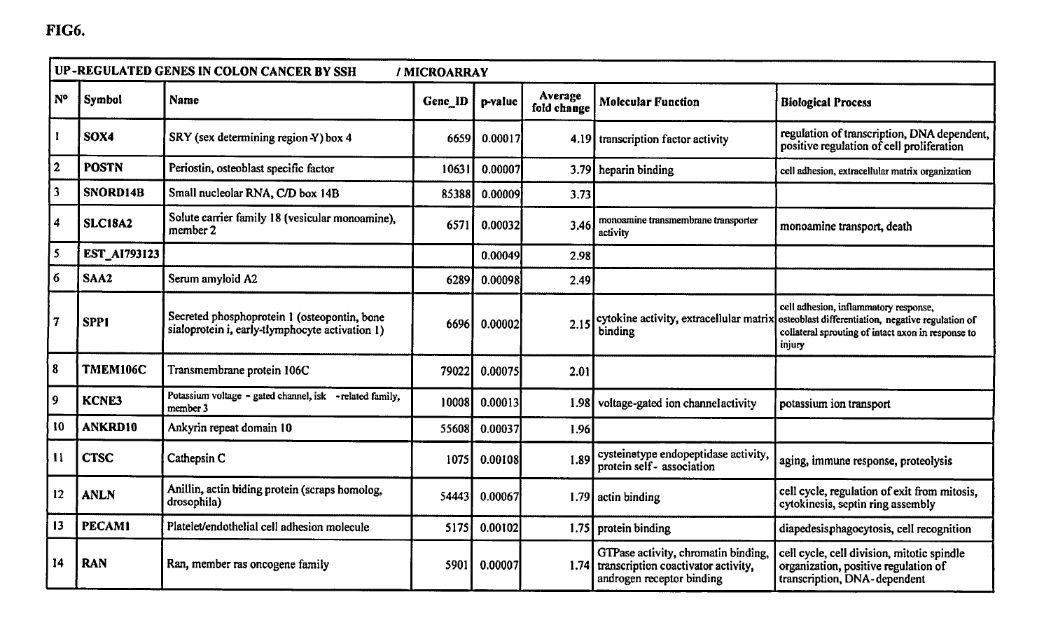

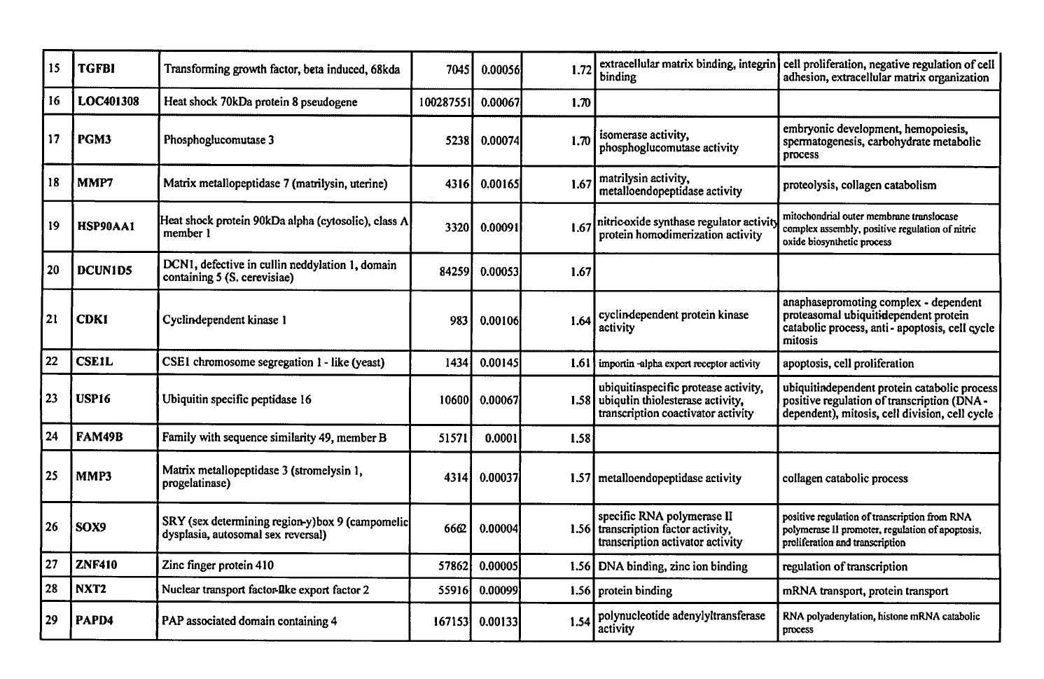

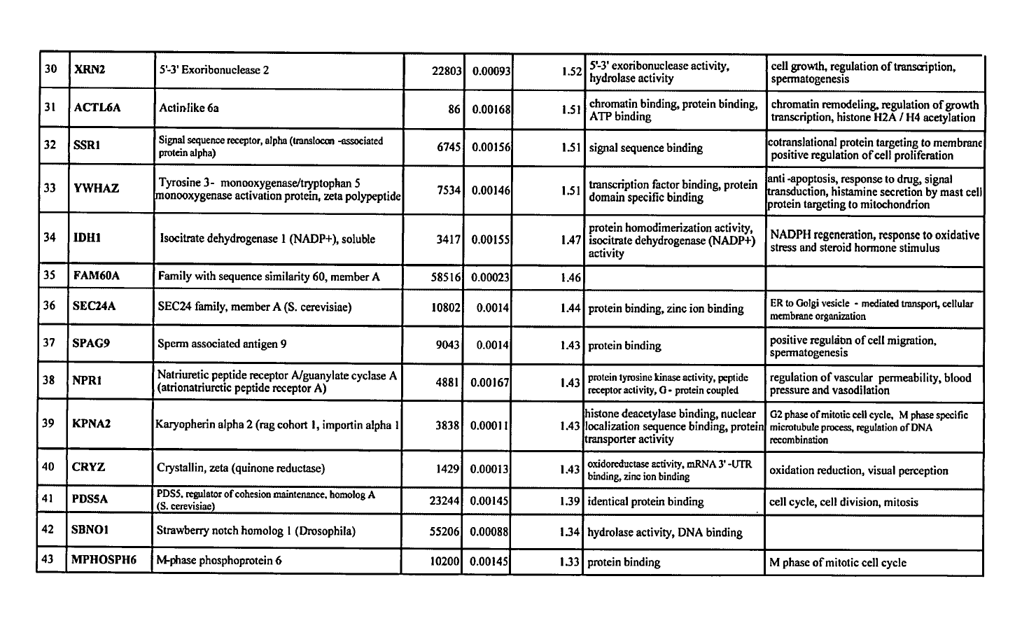

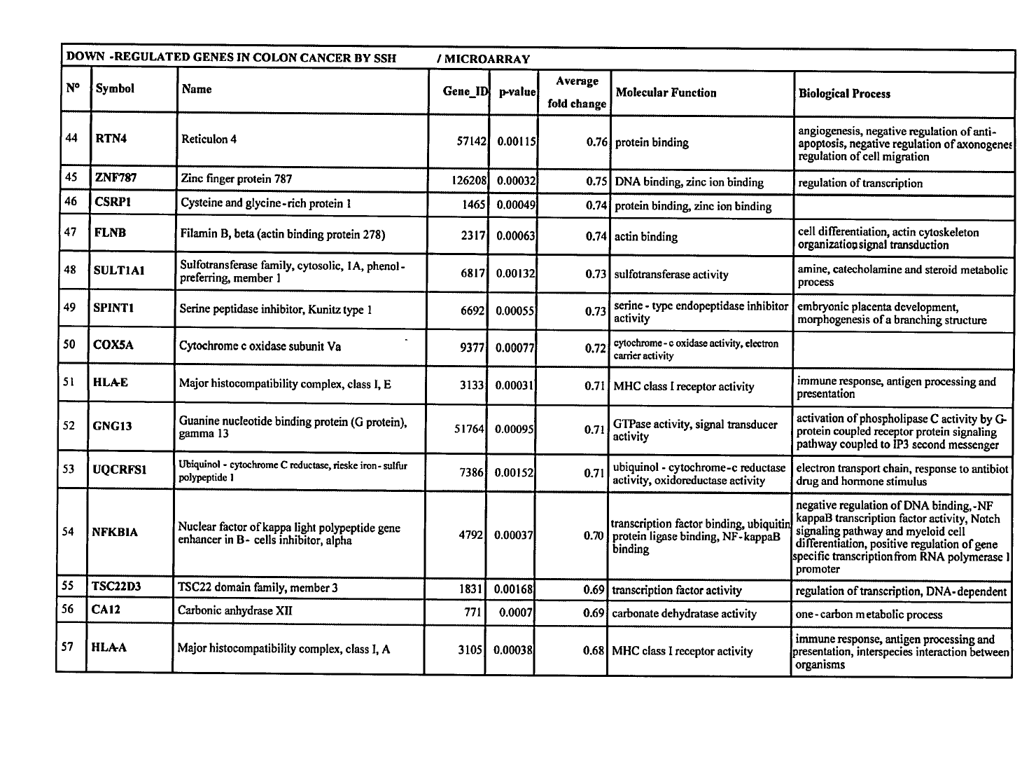

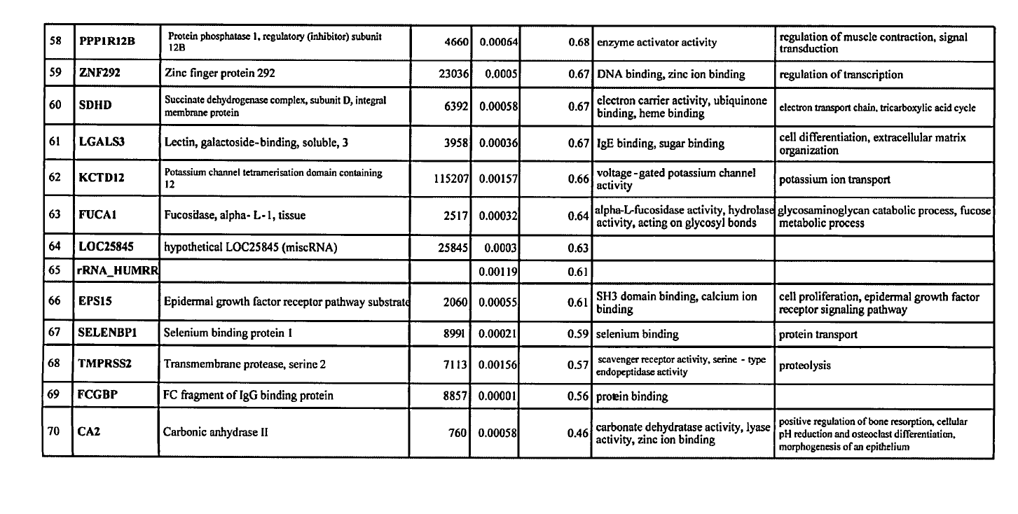

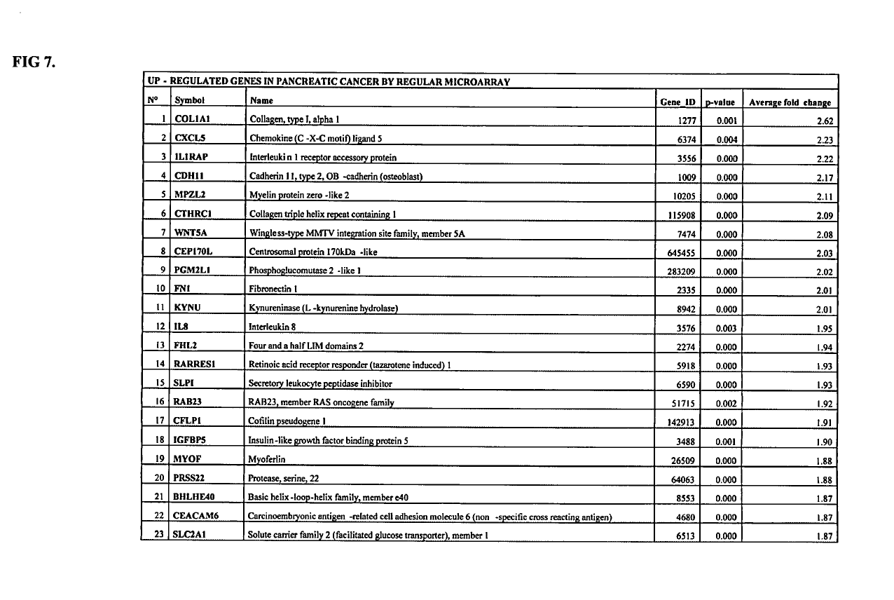

FIG. 7 shows the differential genes obtained using the regular microarray-strategy. The fold change of the tumor/adjacent ratio (T/A) and the statistical significance (p-value) are included.

16 genes were selected in order to validate the data obtained through the regular microarray strategy (SPARC, PDGFRB, TREM2, BHLHE40, TOP2A, CALU, SBNO2, PMEPA1, IGFBP5, CTSK, CDH11, COL4A1, TGFBI, PCK2, SNAI2 and FN1), which were quantified using real-time PCR.

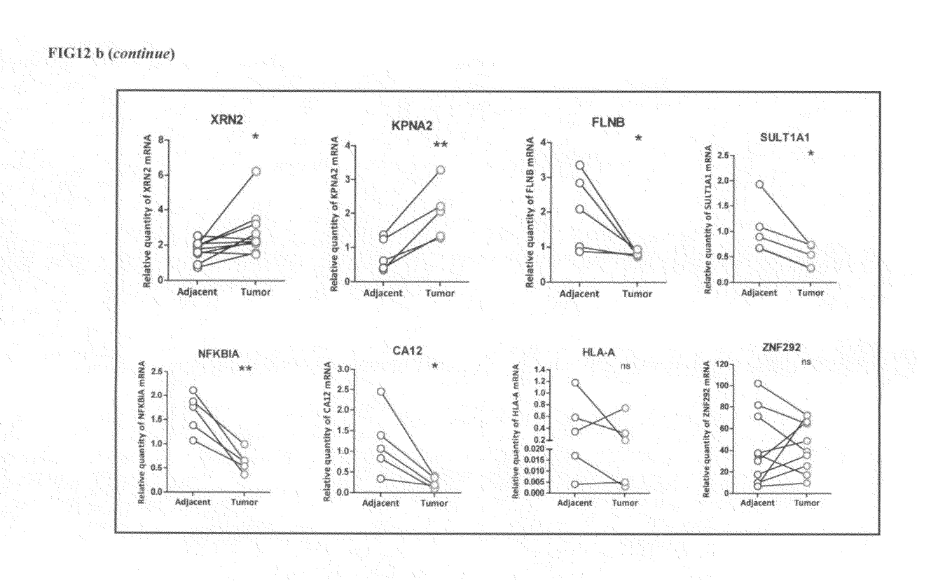

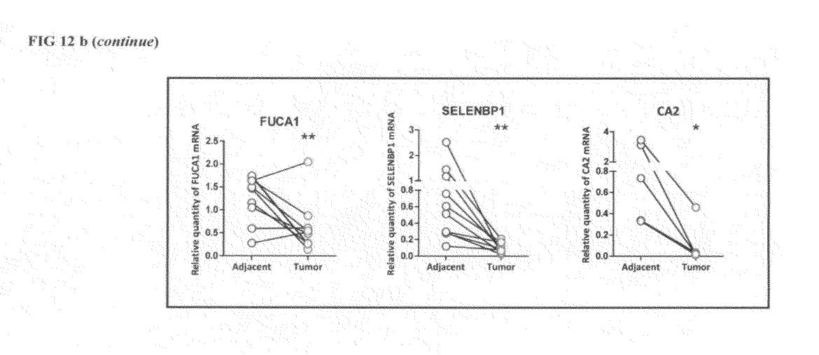

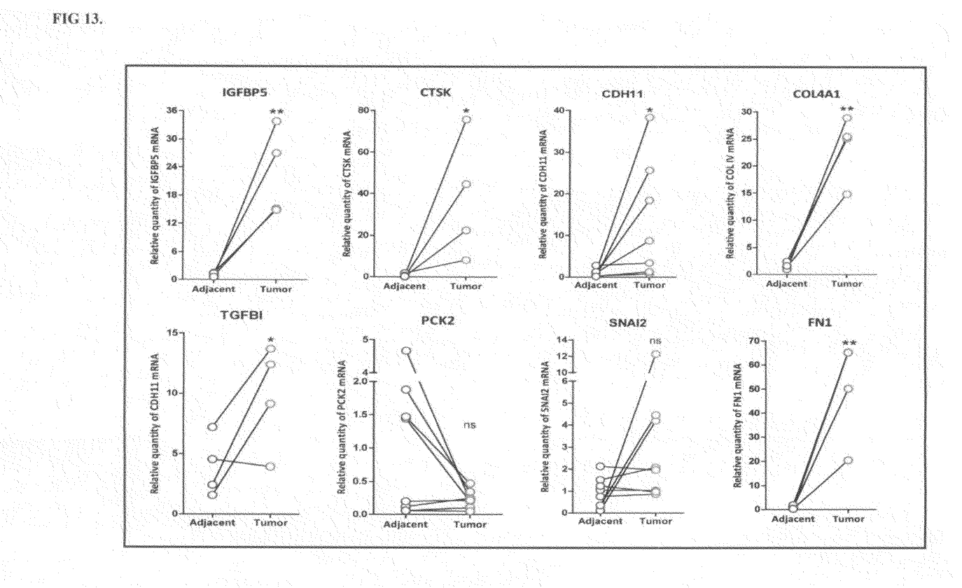

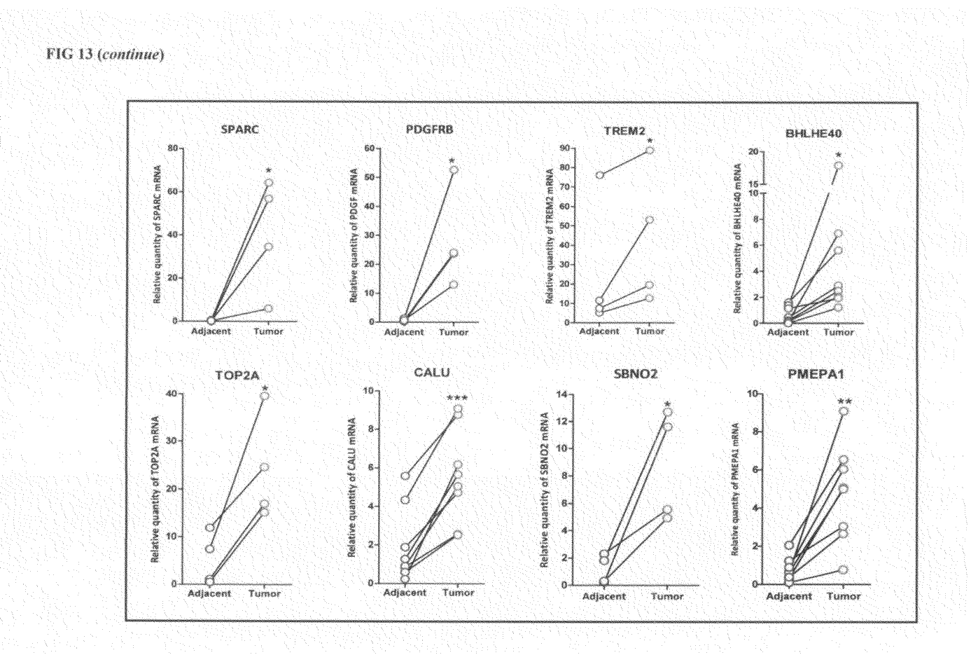

The differential expression was confirmed in 14 genes (p<0.05) and in 2 cases they showed the same expression trend (PCK2 and SNAI2), but not statistically significant. In conclusion, 87.5% of the genes studied were validated (FIG. 13).

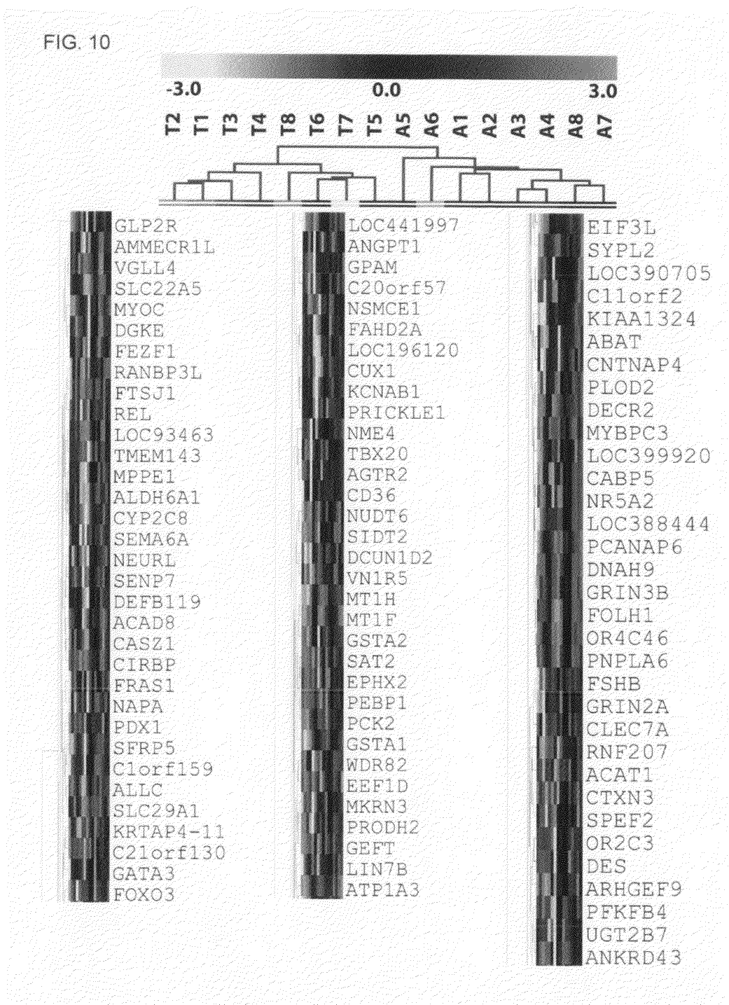

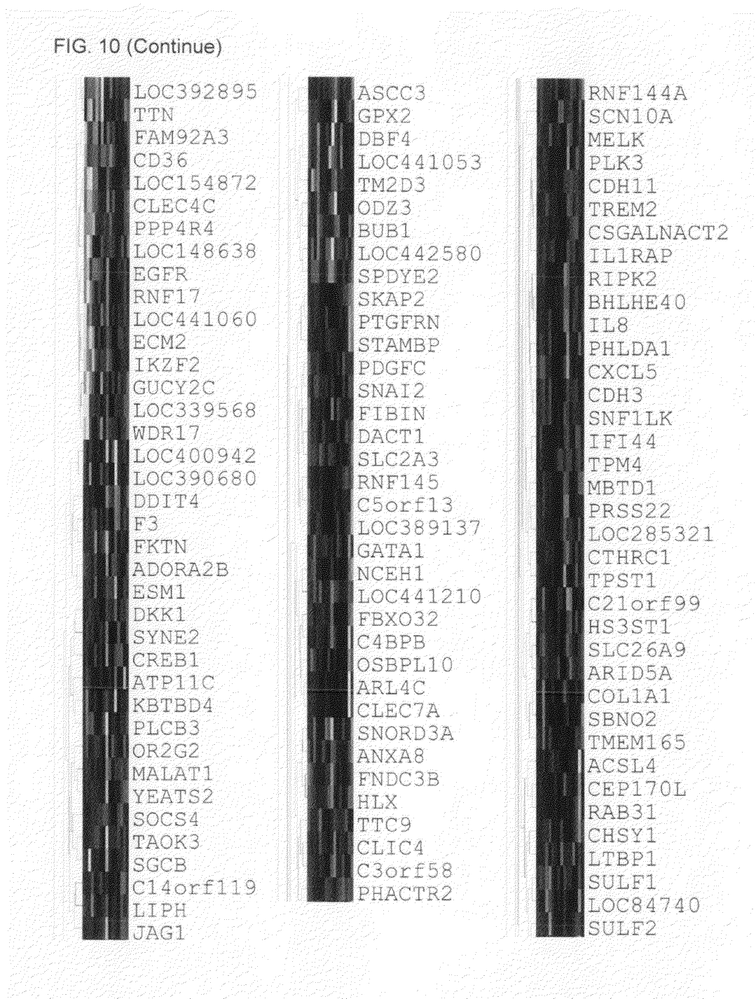

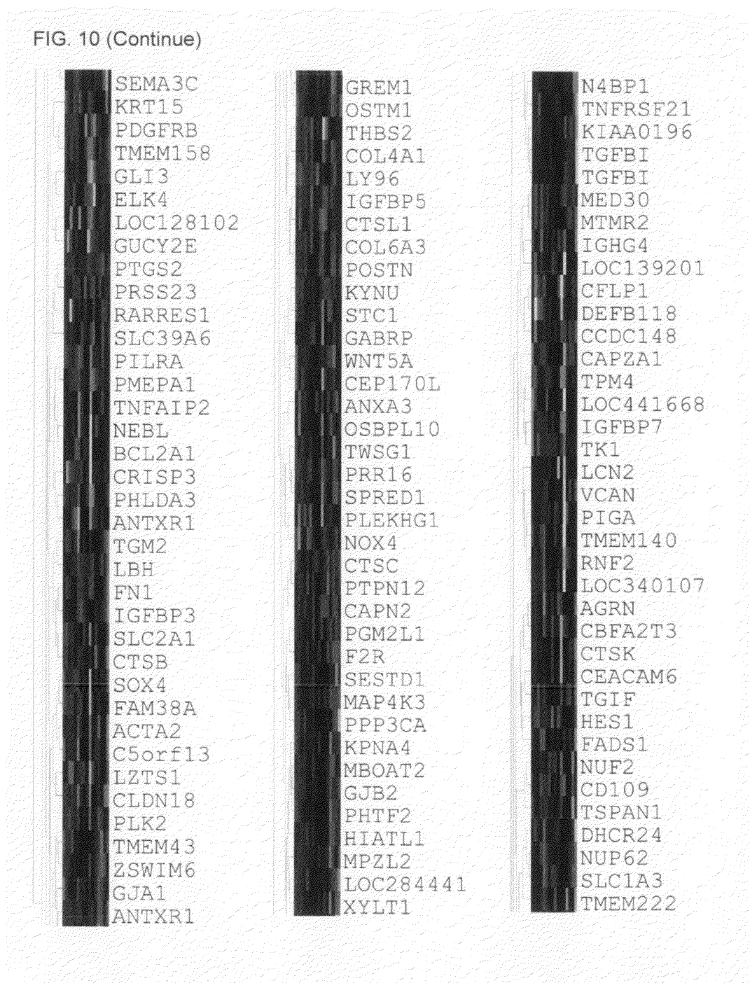

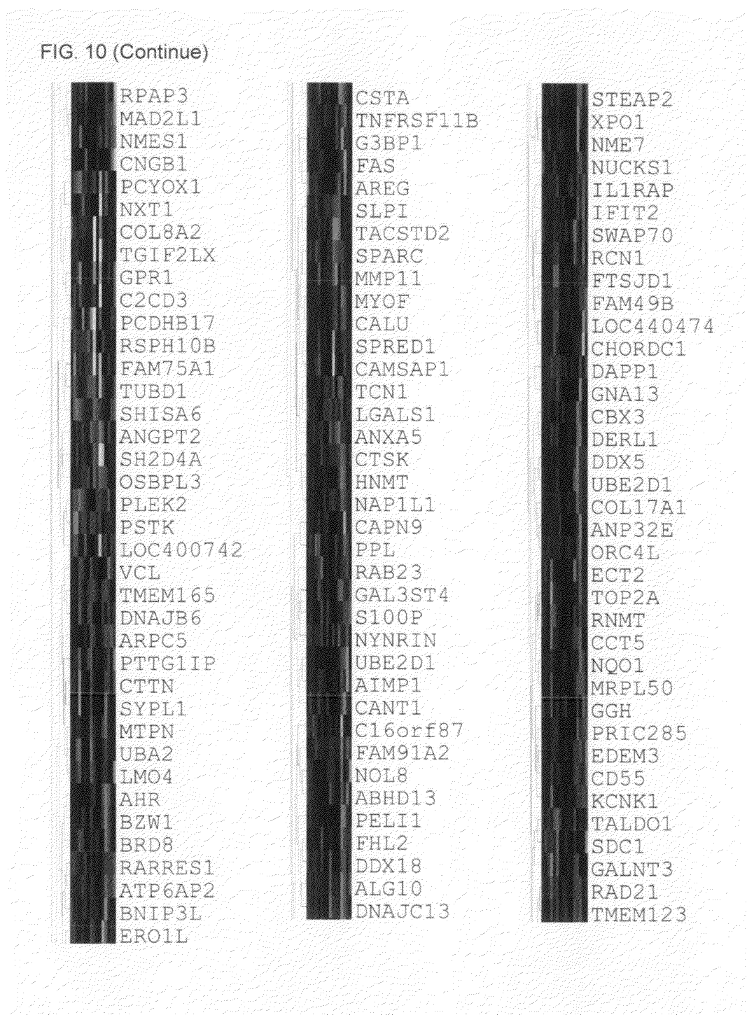

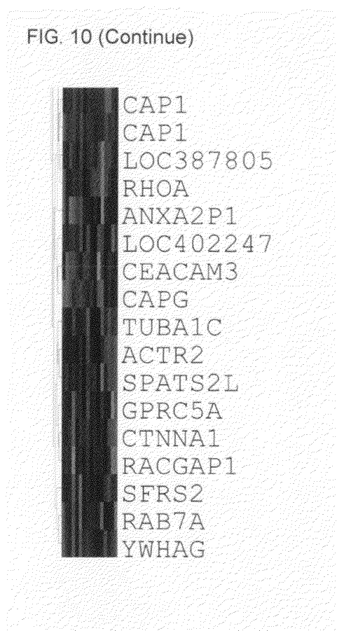

An unsupervised hierarchical clustering of the normalized “M” values (M=Log₂ red channel intensity/green channel intensity) of each differential gene and each of the cases studied showed that the genes obtained from the direct strategy are capable of segregating pancreatic samples into two clusters: Group A includes pancreatic cancer samples and Group B the non-cancerous adjacent tissue New therapeutic targets for gastric, colon and pancreatic cancer (FIG. 10).

### Example 4

The new genes selected in this study are considered new therapeutic targets of the disease. Up-regulated expression of specific genes could be targeted with therapeutic antagonists such as small interfering RNA (siRNA), antibodies for the over-expressed sequences or other modulators (such as inhibitors or antagonists that alter the interaction between the over-expressed polypeptide and its binding site). Genes with a decrease in the expression levels or biological activity could be treated therapeutically so as to increase their activity, for example, by incorporating a sequence of nucleic acids in the cell that encode for under-expressed sequences or using agonists that increase their bioavailability.

Therapeutic methods include introduction of an agent that modulates the activity of the deregulated gene. Specifically, the interfering RNA inhibits gene transcription and translation and promotes the degradation of the mRNA or inhibits the expression of the protein that encodes for the gene product.

We selected siRNA strategies to target 9 over-expressed genes that emerged from this study using specific cell lines of each type of cancer. These genes are: RCC2, DUSP14 and NEK2 for gastric cancer, JPH1, PPAT and GTPBP4 for colon cancer, and CALU, PMEPAI and BHLHE40 for pancreatic cancer.

RCC2 (regulator of chromosome condensation -2), also known as TD-60, has been characterized as a member of the chromosome passenger protein family that plays a role in the control of cytokinesis in mammalian cells. RCC2 is required for alignment of the chromosomes in the mitotic spindle, for centromeric targeting of the chromosomes passenger complex (CPC) and for activation of Aurora-B kinase, the catalytic subunit of CPC (Rosasco-Nitcher, Lan et al. 2008). It has been specifically demonstrated that RCC2 is required for the progression of the cell cycle from prometaphase to metaphase. RCC2 presents the structural homology to the RCC1 family and it has been reported to bind directly to a nucleotide-free form of Rac1 and act as a Rac1 guanine nucleotide exchange factor (GEF) (Mollinari, Reynaud et al. 2003). On the other hand, studies based on large-scale mapping of protein-protein interaction identified the association of RCC2 with Arf6 (Ewing, Chu et al. 2007). RCC2 is currently described to participate in the fibronectin-dependent adhesion signaling pathway to restrict the activation of Rac1 and Arf6 and it is a regulator of the formation of the adhesion complex, cell diffusion and directional migration (Humphries, Byron et al. 2009).

DUSP-14 (dual specificity phosphatase 14), also known as MPK-6, is an atypical MKP (MAP kinase phosphatase) that is comprised of one single DSP domain (dual specific phosphatase) that is capable of dephosphorylating in serine and threonine. DUSP-14 can dephosphorylate ERK ½ and JNK in vitro. DUSP-14 has been described to interact with CD28 by T-cell receptor (TCR) induction and CD28 co-stimulation in T cells, suggesting that DUSP-14 works as a negative-feedback regulator of the CD28 co-stimulating signal (Marti, Krause et al. 2001) (Kondoh and Nishida 2007).

NEK 2 (NIMA (never-in-mitosis gene a)-related kinase 2) is a serine/threonine kinase involved in the regulation of the cell cycle (Fry, Mayor et al. 1998). The specific function of Nek2 is to start the separation of the centrosomes in G2/M by phosphorylation of two components of the intercentriolar linkage, rootletin and C-Nap1 (Fry, Mayor et al. 1998; Bahe, Stierhof et al. 2005; Yang, Adamian et al. 2006). Nek2 has been over-expressed in several cancer cell lines and the primary lines of breast cancer and it has been identified as a potential target for colangiocarcinoma and a new molecular therapy target in breast carcinoma (Kokuryo, Senga et al. 2007; Tsunoda, Kokuryo et al. 2009).

PMEPA1 (prostate transmembrane protein, androgen induced 1) specifically inhibits the TGF-β/Activin signaling pathway by disturbing the recruitment of Smad2 and Smad3 by SARA in the TβRI receptor (Watanabe, Itoh et al. 2010). Elevated levels of PMEPA1 have been observed in stomach, rectum and prostate tumor tissues (Rae, Hooper et al. 2001; Xu, Shi et al. 2003; Sheffer, Bacolod et al. 2009). Alterations in the signaling pathway of PMEPA1, such as hereditary mutations, somatic mutations and an abnormal expression of components in this pathway, have been frequently associated with the malignant process. Considering this information, PMEPA1 could become a therapeutic target.

BHLHE40 (basic helix-loop-helix family, member e40) is a transcription factor that has been found to be up-regulated in certain types of cancer like breast cancer (Currie, Hanrahan et al. 2004) and gastric cancer (Zheng, Jia et al. 2009). The expression of BHLHE40 has been induced by TGF-β(Ehata, Hanyu et al. 2007) and hypoxia (Miyazaki, Kawamoto et al. 2002). BHLHE40 has been recently associated with the differentiation of glioma cancer, a phenomenon associated with the aggressiveness of this type of tumor (Carro, Lim et al. 2010). BHLHE40 has the potential to become a therapeutic target.

CALU has been identified as an endoplasmic reticulum protein and it acts as an inhibitor of γ-carboxylation dependent on vitamin K during the synthesis of coagulation factors (Wajih, Sane et al. 2004; Wajih, Owen et al. 2008). Extracellular calumenin has been associated with modifications of the cytoskeleton and the cell cycle using a potential paracrine or autocrine mechanism (Ostergaard, Hansen et al. 2006). This molecule has been associated with the secretion path and can be found in endoplasmic reticulum and in the Golgi apparatus, secreted to the extracellular spaces of cells in culture (Vorum, Hager et al. 1999). The relationship between CALU and tumor processes has not been studied in detail. An over-expression of the calumenin protein has been found in cancer cells from the cervix that have acquired a resistance to cysplatin (Castagna, Antonioli et al. 2004) and in fibroblasts and transformed HeLa cells (Karlmark, Freilinger et al. 2005; Mazzucchelli, Gabelica et al. 2008). CALU has the potential to become a therapeutic target.

PPAT protein is a member of the purine/pyrimidine phosphoribosyltransferase family. This protein is a regulatory allosteric enzyme that catalyzes the first step of de novo purine nucleotide biosynthesis. This gene and PAICS/AIRC, a bifunctional enzyme catalyzing steps six and seven in the purine nucleotide biosynthesis pathway, are located in close proximity on chromosome 4 (provided by RefSeq Pubmed).

GTPBP4 is a GTPase that function as molecular switches that can flip between two states: active, when GTP is bound, and inactive, when GDP is bound. ‘Active’ in this context usually means that the molecule acts as a signal to trigger other events in the cell. When an extracellular ligand binds to a G-protein-linked receptor, the receptor changes its conformation and switches on the trimeric G proteins that associate with it by causing them to eject their GDP and replace it with GTP. The switch is turned off when the G protein hydrolyzes its own bound GTP, converting it back to GDP. But before that occurs, the active protein has an opportunity to diffuse away from the receptor and deliver its message for a prolonged period to its downstream target (provided by RefSeq Pubmed).

JPH1 The protein encoded by this gene is a component of junctional complexes and is composed of a C-terminal hydrophobic segment spanning the endoplasmic/sarcoplasmic reticulum membrane and a remaining cytoplasmic domain that shows specific affinity for the plasma membrane. This gene is a member of the junctophilin gene family (provided by RefSeq Pubmed).

We initially performed an analysis of the efficiency of inhibition of 6 selected siRNAs by qRT-PCR.

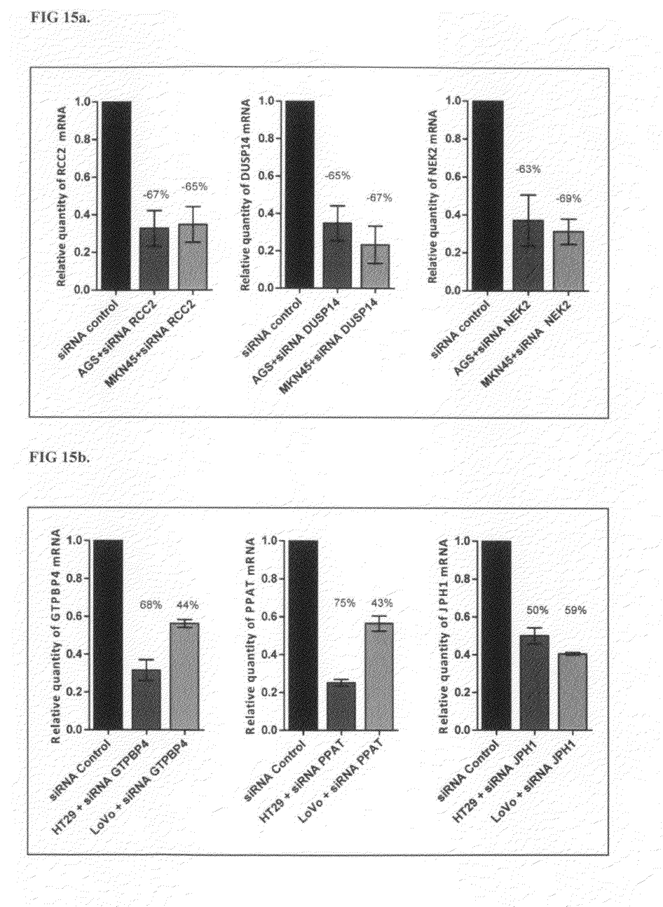

mRNA levels of RCC2, DUSP14 and NEK2 was studied in the AGS and MKN45 gastric adenocarcinoma cell lines 48 hours after transfection with 5 nM of each siRNA. After treatment, the expression of RCC2 mRNA was reduced 67.1%±16.5 in AGS and 65.2%±13.3 in MKN45 cells. The reduction for DUSP14mRNA was 65.3%±13.3 in AGS and 76.7%±14.1 in MKN45. For NEK2 mRNA, the reduction was of 62.9%±19.0 in AGS and 68.8%±9.5 in MKN45 (FIG. 15a). Data is expressed as percentage of control transfected with scramble siRNA.

The inhibition of the expression of JPH1, PPAT and GTPBP4 was studied in HT29 and LoVo colon cancer cells in a way similar to that of the gastric cancer cell lines. For GTPBP4, a decrease of 68% and 44% was obtained in HT29 and LoVo cells respectively. For PPAT, a decrease of 75% and 43% was observed in HT29 and LoVo cells respectively and for JPH1, a decrease of 50% and 59% was observed in HT29 and LoVo cells respectively (FIG. 15b).

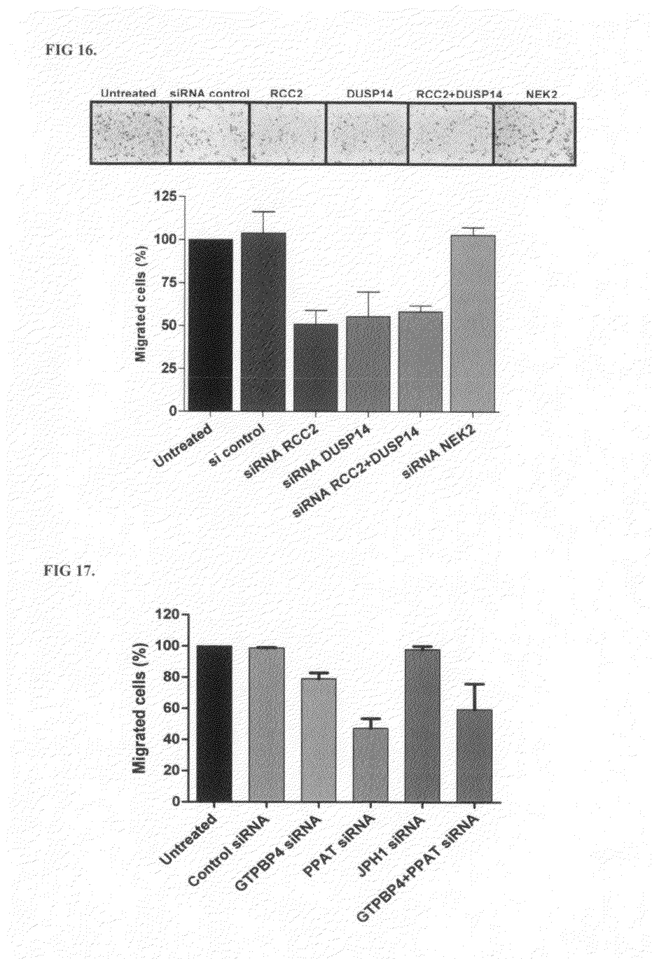

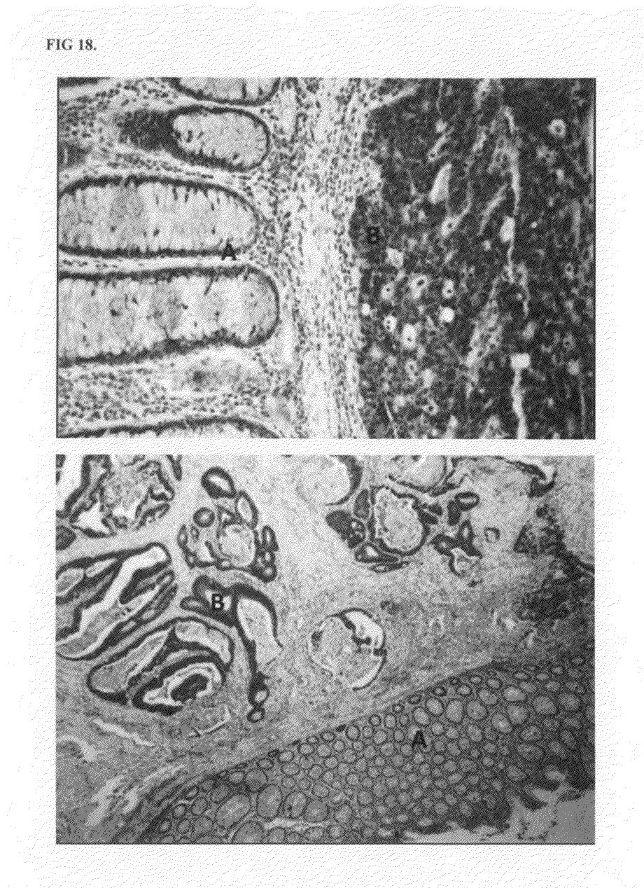

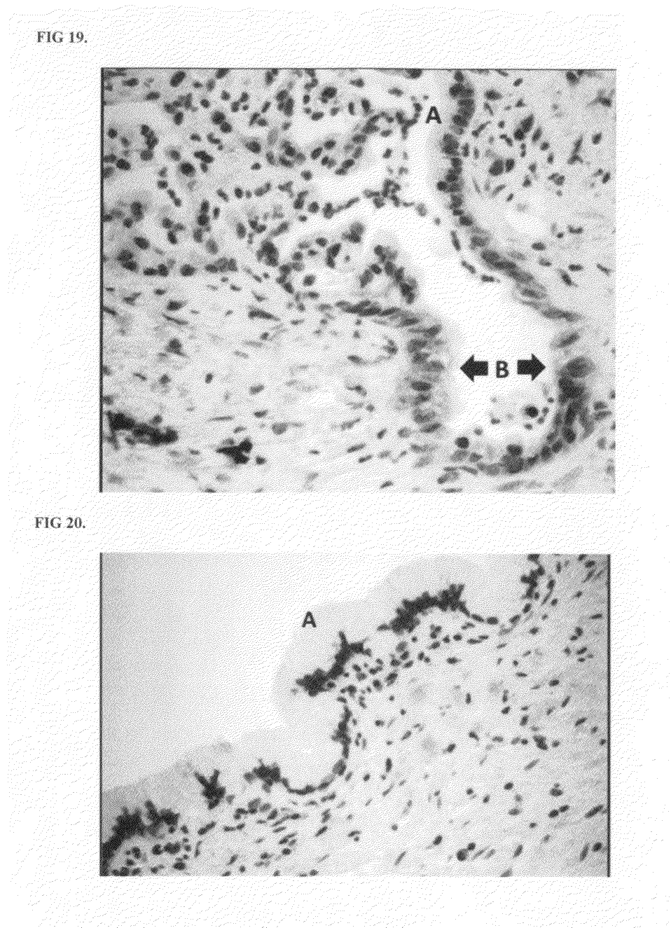

We also evaluated the migratory capacity of AGS transfected with each siRNA. A comparison of the effect of each siRNA compared to the control revealed that the RCC2 and DUSP 14 siRNA reduced the migration capacity of AGS cells by 51% and 55%, respectively. The migration capacity after transfecting the cells with both RCC2 and DUSP14 siRNA was 58%. Cells transfected with control siRNA and with NEK2 siRNA exhibited no differences compared to control cells (FIG. 16).

In the colon cancer cells, a reduction was seen in the migratory capacity for PPAT1 siRNA and its combination with GTBP4 compared to the control (FIG. 17).

These data suggest that these genes can be considered good candidates for therapeutic intervention using interference RNA because it is possible to reduce cell migration, a central event in metastasis.

### Example 5

### Selection of Genes that Encode for Secreted Proteins as Potential Biomarkers

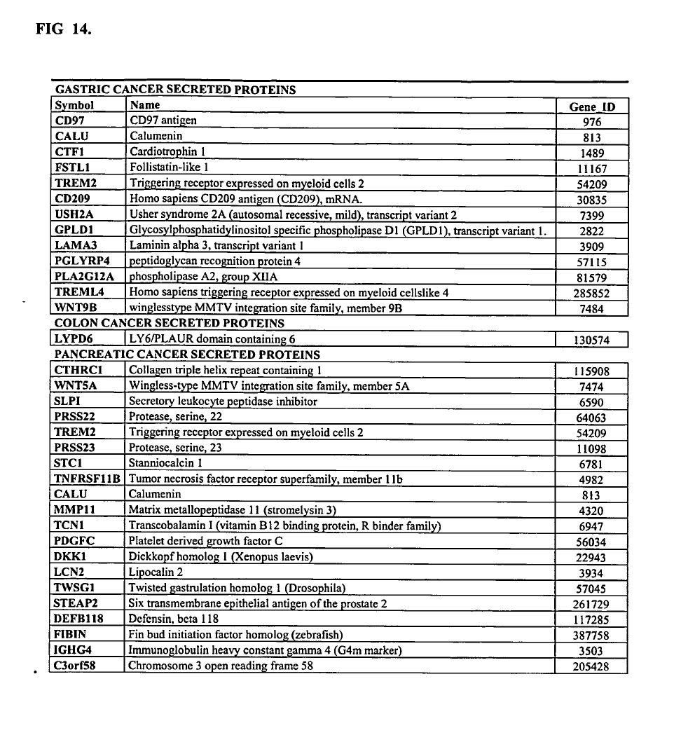

It is known that genetic disorders during malignant transformation lead to the appearance of autocrine and endocrine stimulators, with the consequent over-expression of secreted proteins such as growth factors, cytokines and hormones (Welsh, Sapinoso et al. 2003). Therefore, it is relevant to study new protein markers for the diagnosis of cancer and potential future therapeutic intervention. We combined the differential expression profile obtained for each type of cancer with algorithms to predict candidate genes that encode for secreted proteins. The search was done in Gene Ontology (GO), using the biotechnology resource of DAVID. Among the differentially expressed genes for gastric cancer, 29 genes were identified that encode for secreted proteins: TREM-2, FSTL1, CCL3, APOC-1, CALU, IGFBP7, ASPN, LUM, TGFB1, SPARC, CD209, FCGBP, USH2A, AGR2, GKN1, GKN2, GPLD1, IL8BP, LAMA3, PGLYRP4, PLA2G12A, PLA2G12B, PDG4, PGC, REG3A, TFF1, TFF2, TREML4, WNT9B. 11 genes for colon cancer were identified that en code for secreted proteins: SPP1, MPP7, TGFB1, LYPD6, COL5A2, CALU, FCGBP, IL8, GSN. For pancreatic cancer, at least 20 genes were identified that encode for secreted proteins: CTHRC1, TCN1, PRSS22, PRSS23, DKK1, MMP11, CALU, SLPI, STC1, WNT5A, FIBIN, IGHG4, TWSG1, STEAP2, PDGFC, TNFRSF11B, TREM2, DEFB118, LCN2 and C3orf58 (FIG. 14).

We also want to highlight 11 genes whose products are secreted. The result of the Regular strategy includes IL8 and GSN. While the result of the strategy SSH/Microarray includes; POSTN, SAA2, SPP1, MMP3, SPINT1, TMPRSS2 and FCGBP. MMP7 and TGFBI were detected by both strategies. It is important to consider these genes as potential tumor markers helpful in the diagnosis, prognosis or monitoring of the disease.

Secreted proteins are potential biomarkers of these gastrointestinal cancers. An analysis of these proteins individually or collectively through antibodies could be done in easily accessible biological fluids such as plasma or serum and in fluids obtained from more invasive clinical procedures, such as peritoneal fluid, gastric juice or pancreatic juice.

What is claimed is:

1. A method to identify targets for cancer treatment, said method comprising identifying differently expressing genes in tumor tissue and adjacent healthy tissue by comparing expression of transcripts in SHH-analysis and cDNA micorarray analysis.

2. A method to treat cancer, said method comprising the steps of:
a) identifying new target genes by the method of claim 1;
b) selecting upregulated genes; and
c) modulating over expression of one or more of the selected genes by siRNA, specific inhibitors or antagonists.

3. The method of claim 2, wherein the cancer is gastric cancer, and the upregulated genes are selected from the group consisting of RCC2, DUSP14, and NEK2.

4. The method of claim 2, wherein the cancer is colon cancer, and the upregulated genes are selected from the group consisting of JPH1, RPAT and GTPBP4.

5. The method of claim 2, wherein the cancer is pancreatic adenocarcinoma, and the upregulated genes are selected from the group consisting of CALU, PMEPA1 and BHLHE40.

6. A method to treat cancer, said method comprising the steps of:
a) identifying new target genes by the method of claim 2;
b) selecting downregulated genes; and
c) modulating under-expression by increasing bioavailability of under-expressed protein.

7. Novel protein markers for gastric cancer, said markers being selected from the group consisting of: TREM-2, FSTL1, CCL3, APOC-1, CALU, IGFBP7, ASPN, LUM, TGFB1, SPARC, CD209, FCGBP, USH2A, AGR2, GKN1, GKN2, GPLD1, IL8BP, LAMA3, PGLYRP4, PLA2G12A, PLA2G12B, PDG4, PGC, REG3A, TFF1, TFF2, TREML4, and WNT9B.

8. Novel protein markers for colon cancer, said markers being selected from the group consisting of: SPP1, MPP7, TGFB1, LYPD6, COL5A2, CALU, FCGBP, IL8, and GSN.

9. Novel protein markers for pancreatic adenocarcinoma, said markers being selected from the group consisting of: CTHRC1, TCN1, PRSS22, PRSS23, DKK1, MMP11, CALU, SLPI, STC1, WNT5A, FIBIN, IGHG4, TWSG1, STEAP2, PDGFC, TNFRSF11B, TREM2, DEFB118, LCN2, and C3orf58.

10. Novel plasma tumor markers, said markers being selected from the group consisting of IL-8, GSN, POSTN, SAA2, SPP1, MMP3, SPINT1, TMPRSS, FCGBP, MMP7 and TGFbetal.


SEQUENCE LISTING (7 sequences)

SEQ ID NO: 1 - DNA, 32 bp, Homo sapiens
ctaatacgactcactatagggctcgagcggcc

SEQ ID NO: 2 - DNA, 21 bp, Homo sapiens
  PPAT/ misc_RNA (1)..(21): siRNA_QIAGEN
uucaguuaucgcacuuuggta

SEQ ID NO: 3 - DNA, 21 bp, Homo sapiens
  JPH1 / misc_RNA (1)..(21): siRNA_QIAGEN
uuuagcaucuacaccuucctg

SEQ ID NO: 4 - DNA, 21 bp, Homo sapiens
  GTPBP4 / misc_RNA (1)..(21): siRNA_QIAGEN
uucauaucaaacacgugucta

SEQ ID NO: 5 - DNA, 21 bp, Homo sapiens
  DUSP14 / misc_RNA (1)..(21): siRNA_QIAGEN
uaacagcuaaauaauaagggt

SEQ ID NO: 6 - DNA, 21 bp, Homo sapiens
  NEK2 / misc_RNA (1)..(21): siRNA_QIAGEN
uuaaguuuaacauccucguaa

SEQ ID NO: 7 - DNA, 21 bp, Homo sapiens
  RCC2 / misc_RNA (1)..(21): siRNA_QIAGEN
ugcgguaagcagcuugcugtt